(19) United States
(12) Patent Application Publication
(10) Pub. No.: US 2003/0134283 A1
(43) Pub. Date: Jul. 17, 2003

(54) Genes regulated in dendritic cell differentiation

(76) Inventors: David P. Peterson, San Jose, CA (US); Cecilia I. Pearson, Palo Alto, CA (US); Benjamin G. Cocks, Sunnyvale, CA (US)

Correspondence Address: INCYTE GENOMICS, INC., 3160 PORTER DRIVE, PALO ALTO, CA, 94304, US

(21) Appl. No.: 09/971,392

(22) Filed: Oct. 3, 2001

Related U.S. Application Data
(60) Provisional application No. 60/237,652, filed on Oct. 3, 2000.

(51) Int. Cl. C12Q 1/68; C07H 21/04

(52) U.S. Cl. 435/6; 536/23.1

(57) ABSTRACT

The present invention relates to a combination comprising a plurality of cDNAs which are differentially expressed in dendritic cells, which may be used in their entirety or in part to diagnose, to stage, to treat, or to monitor the treatment of a subject with cancer, infectious disease, autoimmunity, allergy, and graft versus host disease or to enhance a vaccine.

### FIELD OF THE INVENTION

The present invention relates to a combination comprising a plurality of cDNAs which are differentially expressed in dendritic cells and which may be used entirely or in part to diagnose, to stage, to treat, or to monitor the progression or treatment of disorders such as cancer, infectious disease, autoimmunity, allergy, and graft versus host disease.

### BACKGROUND OF THE INVENTION

Array technology can provide a simple way to explore the expression of a single polymorphic gene or the expression profile of a large number of related or unrelated genes. When the expression of a single gene is examined, arrays are employed to detect the expression of that specific gene or its variants. When an expression profile is examined, arrays provide a platform for examining which genes are tissue specific, carrying out housekeeping functions, parts of a signaling cascade, or specifically related to a particular genetic predisposition, condition, disease, or disorder.

The potential application of gene expression profiling is particularly relevant to characterizing lineage differences during cellular development that will improve diagnosis, prognosis, and treatment of disease. For example, both the levels and sequences expressed in dendritic cells from subjects with autoimmunity may be compared with the levels and sequences expressed in dendritic cells from normal subjects.

Dendritic cells (DC) are antigen presenting cells (APC) that play a key role in the primary immune response because of their unique ability to present antigens to naive T cells. In addition, DC differentiate into separate subsets that sustain and regulate immune responses following initial contact with antigen. DC subsets include those that preferentially induce particular T helper 1 (Th1) or T helper 2 (Th2) responses and those that regulate B cell responses. Moreover, DC are increasingly being used to manipulate immune responses, either to downregulate an aberrant autoimmune response or to enhance vaccination or a tumor-specific response.

DC are functionally specialized in correlation with their particular differentiation state. CD34+ myeloid cells found in the bone marrow mature in response to as yet unclear signals into CD14+ CD11c+ monocytes. An innate or antigen non-specific response takes place initially when monocytes circulate to nonlymphoid tissues and respond to lipopolysaccharide (LPS), a bacterially-derived mitogen, and viruses. Such direct encounter with antigen causes secretion of pro-inflammatory cytokines that attract and regulate natural killer cells, macrophages, and eosinophils in the first line of defense against invading pathogens. Monocytes then mature into DC, which capture antigen highly efficiently through endocytosis and antigen receptor uptake. Antigen processing and presentation trigger activation and differentiation into mature DC that express MHC class II molecules on the cell surface and efficiently activate T cells, initiating antigen-specific T cell and B cell responses. In turn, T cells activate DC through CD40 ligand-CD40 interactions, which stimulate expression of the costimulatory molecules CD80 and CD86, the latter most potent in amplifying T cell responses. DC interaction via CD40 with T cells also stimulates the production of inflammatory cytokines such as tumor necrosis factor alpha (TNF-α) and interleukin (IL)-1. Engagement of RANK, a member of the TNF receptor family by its ligand, TRANCE, which is expressed on activated T cells, enhances the survival of DC through inhibition of apoptosis, thereby enhancing T cell activation. The maturation and differentiation of monocytes into mature DC links the antigen non-specific innate immune response to the antigen-specific adaptive immune response.

The process by which monocytes differentiate into immature dendritic cells in vivo has not been fully elucidated. Incubation of monocytes with granulocyte-macrophage colony stimulating factor (GM-CSF) and IL4 in vitro yields cells that exhibit functional and morphological characteristics equivalent to immature dendritic cells found in vivo. Moreover, incubation in vitro of immature dendritic cells with TNF-α, CD40 ligand, LPS, or monocyte-conditioned medium yields mature dendritic cells that are potent activators of naive T cells.

The ability to manipulate DC in vitro and their capacity to mount an effective immune response with small numbers of DC and little antigen has led to: 1) potential immunotherapies for diseases such as cancer, AIDS, and infectious diseases, and 2) enhanced vaccine efficacy. Spontaneous remissions of particular cancers such as renal cell carcinomas and melanomas indicate that the immune system can respond to tumor antigens and eliminate tumors. However, tumors escape immune surveillance through a number of means including secretion of IL-10, macrophage colony stimulating factor, IL-6, and vascular endothelial growth factor, all of which inhibit the activity of DC and promote tolerance of tumor tissue. Delivery of tumor antigen-loaded DC to tumors can induce tumor-specific rejection in animal models. Similarly, pathogens can escape immune surveillance by altering antigen processing and presentation pathways or interfering with maturation of antigen presenting cells. Rather than providing resistance, DC can complicate infection by hosting latent viruses such as Kaposi's virus and cytomegalovirus, and producing HIV-1 and measles virus particles. Vaccines against tumors or infectious pathogens could be improved by systemic or local administration of DC loaded with tumor antigens or attenuated viral particles or components, respectively.

The expression of killer-inhibitor regulatory molecules, chemokines, chemokine receptors, and proteinases in DC has been verified through sequencing of ESTs. Continuing this search through the use of microarrays may reveal new lymphocyte-binding and antigen-processing molecules, transmembrane and secretory products, and transcription factors that may help to explain the specialized features of DC and allow manipulation of the immune system.

The present invention provides for a combination comprising a plurality of cDNAs for use in detecting expression of genes encoding proteins in DC. Such a combination satisfies a need in the art in that it can be employed for the improvement of vaccines, in the evaluation of candidates for tissue or organ transplant, and in the diagnosis, prognosis or treatment of cancer, infectious disease, autoimmunity, allergy, and graft versus host disease.

### SUMMARY

The present invention provides a combination comprising a plurality of cDNAs and their complements which are differentially expressed in dendritic cells (DC) and which are selected from SEQ ID NOs:1-260 as presented in the Sequence Listing. In one embodiment, each cDNA is downregulated in DC at least three-fold, SEQ ID NOs:1-29; in another embodiment, each cDNA is upregulated in DC at least three-fold, SEQ ID NOs:30-241; in another embodiment, each cDNA is upregulated in mature DC at least 2.5-fold, SEQ ID NOs:242-257; in yet another embodiment, each cDNA is downregulated in mature DC at least 2.5-fold, SEQ ID NOs:258-260. In one aspect, the combination is useful to characterize monocyte and dendritic cell populations from subjects with disorders such cancer, infectious disease, autoimmunity, allergy, and graft versus host disease. In a second aspect, the combination is useful in evaluation of subjects prior to tissue or organ transplant. In a third aspect, combination is useful in the diagnosis, progression, treatment or monitoring the treatment of cancer, infectious disease, autoimmunity, allergy, and graft versus host disease. In a fourth aspect, the combination is useful to enhance or to measure enhancement of dendritic cell function during immunotherapy of cancer or other disorders. In a fifth aspect, the combination is useful to measure suppression of dendritic cell function during immunotherapy. In a sixth aspect, the combination is useful to enhance the efficacy of vaccines. In a seventh aspect, the combination is immobilized on a substrate.

The invention provides a high throughput method to detect expression of a nucleic acid which is complementary at least one of the cDNAs of the combination in a sample. The method comprises hybridizing a substrate containing the combination with a sample containing nucleic acids under conditions to form at least one hybridization complex and detecting hybridization complex formation, wherein complex formation indicates expression of at least one complementary nucleic acid in the sample. In one aspect, the sample is from a subject with cancer and differential expression when compared to a standard determines the state of the immune response to the tumor. In another aspect, the sample is from a subject with cancer being treated with dendritic cell immunotherapy, and expression when compared to a standard determines efficacy of the treatment.

The invention also provides a high throughput method of screening a library or plurality of molecules or compounds to identify a ligand. The method comprises combining the combination with a library or a plurality of molecules or compounds under conditions to allow specific binding and detecting specific binding, thereby identifying a ligand. The library or plurality of molecules or compounds are selected from aptamers, DNA molecules, RNA molecules, peptide nucleic acid molecules, mimetics, peptides, transcription factors, repressors, and other regulatory proteins. The invention additionally provides a method for purifying a ligand, the method comprising combining a cDNA of the invention with a sample under conditions which allow specific binding, recovering the bound cDNA, and separating the cDNA from the ligand, thereby obtaining purified ligand.

The invention further provides an isolated cDNA selected from SEQ ID NOs:24-29, 187-202, 205-213, 216-241, 256, 257, 259, and 260 as presented in the Sequence Listing. The invention also provides a vector comprising the cDNA, a host cell comprising the vector, and a method for using a host cell to produce a protein comprising culturing the host cell under conditions for the expression of a protein and recovering the protein from the culture.

The present invention provides a purified protein encoded and produced by a cDNA of the invention. The invention also provides a high-throughput method for using a protein to screen a library or a plurality of molecules or compounds to identify a ligand. The method comprises combining the protein or a portion thereof with the library or plurality of molecules or compounds under conditions to allow specific binding and detecting specific binding, thereby identifying a ligand which specifically binds the protein. The library or plurality of molecules or compounds is selected from aptamers, DNA molecules, RNA molecules, peptide nucleic acid molecules, mimetics, peptides, proteins, agonists, antagonists, antibodies or their fragments, immunoglobulins, inhibitors, drug compounds, and pharmaceutical agents. The invention further provides for using a protein to purify a ligand. The method comprises combining the protein or a portion thereof with a sample under conditions to allow specific binding, recovering the bound protein, and separating the protein from the ligand, thereby obtaining purified ligand. The invention still further provides a composition comprising the protein and a pharmaceutical carrier.

The invention also provides methods for using a protein to prepare and purify polyclonal and monoclonal antibodies which specifically bind the protein. The method for preparing a polyclonal antibody comprises immunizing a animal with protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified polyclonal antibodies. The method for preparing and purifying monoclonal antibodies comprises immunizing a animal with a protein under conditions to elicit an antibody response, isolating antibody producing cells from the animal, fusing the antibody producing cells with immortalized cells in culture to form monoclonal antibody producing hybridoma cells, culturing the hybridoma cells, and isolating from culture monoclonal antibodies which specifically bind the protein.

### DESCRIPTION OF THE SEQUENCE LISTING AND TABLES

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

The Sequence Listing is a compilation of cDNAs obtained by sequencing and extending clone inserts. Each sequence is identified by a sequence identification number (SEQ ID NO) and by a template number (TEMPLATE ID).

Table 1 lists the functional annotation and differential expression of each cDNA that has previously been shown to be upregulated in DC. Columns 1 and 2 show the GenBank identification number (GENBANK ID) and functional annotation (DESCRIPTION), respectively, as determined by BLAST analysis, version 1.4 using default parameters (Altschul (1993) J Mol Evol 36:290-300; Altschul et al. (I 990) J Mol Biol 215:403410) of the cDNA against GenBank (release 117; National Center for Biotechnology Information (NCBI), Bethesda Md.). Column 3 shows the balanced differential expression value(s), BDE, produced by each clone that represented the cDNA in the experiment.

Tables 2-5 list the functional annotation and differential expression of the cDNAs of the present invention. Table 2 lists those cDNAs that are downregulated in DC; Table 3, cDNAs that are upregulated in DC; Table 4, cDNAs that are upregulated in mature DC; and Table 5, cDNAs that are downregulated in mature DC. In Tables 2-5, Columns 1, 2, and 3 show the SEQ ID NO, CLONE ID, and TEMPLATE ID, respectively. Columns 4 and 5 show the GenBank identification number (GENBANK ID) and functional annotation (DESCRIPTION), respectively, as determined by BLAST analysis of the cDNA against GenBank (release 117; NCBI). Column 6 shows the database from which functional annotation came. Column 7 shows the BDE for each clone that represents that cDNA. Of those sequences that are unique, only the SEQ ID NO, CLONE ID, TEMPLATE ID, and BDE are noted. For SEQ ID NOs:201-215, column 8 of Table 3 shows the E-VALUE for the template, as determined by BLASTx analysis.

### DESCRIPTION OF THE INVENTION

Definitions

Definitions

“Array” refers to an ordered arrangement of at least two cDNAs on a substrate. At least one of the cDNAs represents a control or standard sequence, and the other, a cDNA of diagnostic interest. The arrangement of from about two to about 40,000 cDNAs on the substrate assures that the size and signal intensity of each labeled hybridization complex formed between a cDNA and a sample nucleic acid is individually distinguishable.

The “complement” of a nucleic acid molecule of the Sequence Listing refers to a nucleotide sequence which is completely complementary over the full length of the sequence and which will hybridize to the nucleic acid molecule under conditions of high stringency.

A “combination” comprises at least two, alternatively about 10, and up to 292 sequences selected from the group consisting of SEQ ID NOs:1-292 as presented in the Sequence Listing.

“cDNA” refers to a chain of nucleotides, an isolated polynucleotide, a nucleic acid molecule, or any fragment or complement thereof. It may have originated recombinantly or synthetically, be double-stranded or single-stranded, coding and/or noncoding, an exon with, or without, an intron from a genomic DNA molecule, and purified or combined with carbohydrate, lipids, amino acids or inorganic elements or substances. Preferably, the cDNA is from about 400 to about 12000 nucleotides.

The phrase “cDNA encoding a protein” refers to a nucleic acid whose sequence closely aligns with sequences that encode conserved regions, motifs or domains identified by employing analyses well known in the art. These analyses include BLAST (Basic Local Alignment Search Tool; Altschul, supra; Altschul et al. (1993) supra) which provides identity within the conserved region. Brenner et al. (1998; Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner, page 6076, column 2).

“Derivative” refers to a cDNA or a protein that has been subjected to a chemical modification. Derivatization of a cDNA can involve substitution of a nontraditional base such as queosine or of an analog such as hypoxanthine. These substitutions are well known in the art. Derivatization of a protein involves the replacement of a hydrogen by an acetyl, acyl, alkyl, amino, formyl, or morpholino group. Derivative molecules retain the biological activities of the naturally occurring molecules but may confer advantages such as longer lifespan or enhanced activity.

“Differential expression” refers to an increased or upregulated or a decreased or downregulated expression as detected by absence, presence, or at least 2.5-fold change in the amount of transcribed messenger RNA or translated protein in a sample.

“Disorder” refers to conditions, diseases or syndromes which fall into the categories of cancer, infectious disease, autoimmunity, allergy, and graft versus host disease.

“Fragment” refers to a chain of consecutive nucleotides from about 200 to about 700 base pairs in length. Fragments may be used in PCR or hybridization technologies to identify identical or related nucleic acid molecules and in binding assays to screen for a ligand. Nucleic acids and ligands identified in this manner are useful as therapeutics to regulate replication, transcription or translation.

A “hybridization complex” is formed between a cDNA and a nucleic acid of a sample when the purines of one molecule hydrogen bond with the pyrimidines of the complementary molecule, e.g., 5′-A-G-T-C-3′ base pairs with 3′-T-C-A-G-5′. The degree of complementarity and the use of nucleotide analogs affect the efficiency and stringency of hybridization reactions.

“Identity” as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:46734680), or BLAST2 (Altschul et al. (1997) supra). BLAST2 may be used in a standardized and reproducible way to insert gaps in one of the sequences in order to optimize alignment and to achieve a more meaningful comparison between them. “Similarity” as applied to proteins uses the same algorithms but takes into account conservative substitutions of nucleotides or residues.

“Ligand” refers to any agent, molecule, or compound which will bind specifically to a complementary site on a cDNA molecule or fragment thereof or to a protein or an epitope thereof. Such ligands stabilize or modulate the activity of polynucleotides or proteins and may be composed of inorganic or organic substances including nucleic acids, amino acids, carbohydrates, fats, and lipids.

“Oligonucleotide” refers a single stranded molecule from about 18 to about 60 nucleotides in length which may be used in hybridization or amplification technologies or in regulation of replication, transcription or translation. Equivalent terms include amplimer, primer, and oligomer.

“Portion” refers to any part of a protein used for any purpose which retains at least one biological or antigenic characteristic of a native protein, but especially, to an epitope for the screening of ligands or for production of antibodies.

“Post-translational modification” of a protein can involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

“Probe” refers to a cDNA that hybridizes to at least one nucleic acid molecule in a sample. Where targets are single stranded, probes are complementary single strands. Probes can be labeled with reporter molecules for use in hybridization reactions including Southern, northern, in situ, dot blot, array, and like technologies or in screening assays.

“Protein” refers to a polypeptide or any portion thereof. An “oligopeptide” is an amino acid sequence from about five residues to about 15 residues that is used as part of a fusion protein to produce an antibody.

“Purified” refers to any molecule or compound that is separated from its natural environment and is from about 60% free to about 90% free from other components with which it is naturally associated.

“Sample” is used in its broadest sense as containing nucleic acids, proteins, antibodies, and the like. A sample may comprise a bodily fluid; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue or tissue biopsy; a tissue print; a fingerprint, buccal cells, skin, or hair; and the like.

“Specific binding” refers to a special and precise interaction between two molecules which is dependent upon their structure, particularly their molecular side groups. For example, the intercalation of a regulatory protein into the major groove of a DNA molecule, the hydrogen bonding along the backbone between two single stranded nucleic acids, or the binding between an epitope of a protein and an agonist, antagonist, or antibody.

“Substrate” refers to any rigid or semi-rigid support to which cDNAs or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

“Variant” refers to molecules that are recognized variations of a cDNA or a protein encoded by the cDNA. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid.

The Invention

The present invention provides for a combination comprising a plurality of cDNAs or their complements, SEQ ID NOs:1-260, which can be used on a substrate to diagnose, to stage, to treat or to monitor the progression or treatment of a disorder such as cancer, infectious disease, autoimmunity, allergy, and graft versus host disease. These cDNAs represent known and novel genes differentially expressed in DC. The combination is used in its entirety or in part, as subsets of cDNAs downregulated in immature DC, SEQ ID NOs:1-29; cDNAs upregulated in immature DC, SEQ ID NOs:30-241; cDNAs upregulated in mature DC, SEQ ID NOs:242-257; or cDNAs downregulated in mature DC, SEQ ID NOs:258-260. SEQ ID NOs:24-29 represent novel cDNAs downregulated in immature DC; SEQ ID NOs:187-202, 205-213 and 216-241 represent novel cDNAs upregulated in immature DC; SEQ ID NOs:256 and 257 represent novel cDNAs upregulated in mature DC; and SEQ ID NOs:259 and 260 represent novel cDNAs downregulated in mature DC. Since the novel cDNAs were identified solely by their differential expression, it is not essential to know a priori the name, structure, or function of the gene or its encoded protein. The usefulness of the cDNAs exists in their immediate value as diagnostics.

In one embodiment, the combination is useful to characterize monocyte and dendritic cell populations from subjects with disorders such cancer, infectious disease, autoimmunity, allergy, and graft versus host disease. In a second embodiment, the combination is useful in evaluation of subjects prior to tissue or organ transplant. In a third embodiment, the combination is useful in the diagnosis, progression, treatment or monitoring the treatment of cancer, infectious disease, autoimmunity, allergy, and graft versus host disease. In a fourth embodiment, the combination is useful to enhance or to measure enhancement of dendritic cell function during immunotherapy of cancer or other disorders. In a fifth embodiment, the combination is useful to measure suppression of dendritic cell function during immunotherapy. In a sixth embodiment, the combination is useful to enhance the efficacy of vaccines. In a seventh embodiment, the combination is immobilized on a substrate.

Table 1 shows cDNAs that are upregulated in dendritic cells greater than three-fold and which have been previously shown in the literature to be expressed in dendritic cells. The types of cDNAs that are upregulated in concert during the transition from monocyte to dendritic cell reflect newly acquired DC functions, such as antigen uptake. Both Fc receptors and complement receptors which bind and endocytose antibody-antigen complexes are upregulated on DC. Heat shock proteins, which assist in the capture and transport of antigen, are upregulated approximately five-fold. Molecules involved in the formation of vesicles during endocytosis are upregulated, such as clathrin and clathrin-associated molecules. Fibronectin, which is upregulated up to 80-fold in dendritic cells, can enhance cell adhesion, migration, and proliferation. A large number of cDNAs encoding proteins that are involved in antigen processing and presentation are also upregulated in dendritic cells. These cDNAs include major histocompatibility complex (MHC) class II cDNAs, which encode proteins that present antigen to T cells; cDNAs encoding invariant chain, which is essential for assembly and antigen binding to MHC class II proteins; and cathepsin cDNAs, which encode proteases that reside in lysosomes and degrade antigen into peptides suitable for binding to MHC proteins. In addition, cDNAs encoding chemokines, which attract other immune cells, such as T cells and eosinophils, are upregulated in dendritic cells. Chemokines such as monocyte chemoattractant proteins 1, 2, and 4; eotaxin; thymus- and activation-regulated chemokine; macrophage inflammatory protein-1 alpha; and pulmonary and activation-regulated chemokine that attract both Th1 and Th2 type cells were upregulated, suggesting that immature DC retain their capacity to initiate different types of immune responses and have not yet acquired specialized functionality.

A number of cDNAs are downregulated three to 10-fold in DC relative to monocytes as shown in Table 2. Since these cDNAs include those that have been shown to be expressed in monocytes, but not in immature DC, the cDNAs shown in Table 2 probably have roles in monocyte function. Previously described monocyte-specific cDNAs include SEQ ID NO:4 and 5. These are cDNA variants that encode CL100 protein tyrosine phosphatase, which can suppress MAP kinase protection from apoptosis. Monocytes also express a number of cDNAs that are upregulated in cells stimulated with mitogen, such as those that regulate entry into the G₀/G₁ switch in the cell cycle. These cDNAs include SEQ ID NO:8, which encodes the cellular oncogene cfos, SEQ ID NO:11, which encodes GOS2 protein, and SEQ ID NOs:12 and 13, which are two variant cDNAs that encode a helix-loop-helix phosphoprotein (GOS8) involved in regulating G protein signaling.

Table 2 also shows cDNAs that have not previously been shown to be upregulated in monocytes. For example, two other phosphatases, SEQ ID NOs:9 and 14, are preferentially expressed in monocytes, and together with SEQ ID NOs:4 and 5 are involved in regulating kinases. Several clones representing pre-B cell enhancing factor (PBEF) and its 3′ UTR are upregulated three to six-fold over expression in dendritic cells (SEQ ID NOs:19-25). PBEF is expressed mainly in the bone marrow, liver, and muscle. Expression in bone marrow suggests involvement in the early differentiation stages of hematopoietic cells. The 3′ region of PBEF cDNA contains multiple TATT motifs, which are RNA destabilization sequences found in other upregulated genes including cfos, some of the genes regulating the G₀/G₁ switch in the cell cycle, and other cytokines. PBEF plays a role in the innate immune response by monocytes, since a PBEF homolog is upregulated in the innate immune response of sponges upon contact with xenogeneic sponge tissue. In the immune system, 29% of PBEF transcripts are found in libraries derived from activated granulocytes, monocytes, macrophages, and dendritic cells. Another indication that monocytes are involved in the innate immune response is the expression of a cDNA encoding a chemokine, SCM-1 beta (SEQ ID NO:7) that is constitutively expressed in natural killer cells.

SEQ ID NOs:26-29 are unique cDNAs. SEQ ID NO:26 is upregulated greater than three-fold and encodes a protein about 30% identical to CG9591, an uncharacterized protein expressed in Drosophila melanogaster. SEQ ID NO:26 encodes a protein at least 1019 amino acids long and contains potential protein kinase C and casein kinase II phosphorylation sites and myristylation sites. SEQ ID NOs:27-29 are upregulated between four and six-fold. The occurrence of SEQ ID NO:28 primarily in hemic/immune libraries, 33% of which are derived from macrophages or monocytes, suggests that its function is immune-specific, and in particular, specific to the functions of macrophages and monocytes.

SEQ ID NOs:30-51 are cDNAs that are upregulated in DC and associated with endocytosis, antigen uptake and presentation, and proteolytic processing. SEQ ID NOs:30-32 are cDNAs upregulated about three to five-fold and encode sorting nexins 1-3, which belong to a family of hydrophilic molecules and are partially associated with the cellular membrane. In the immune system, 50% of sequences matching to SEQ ID NO:32 are found in cells of myeloid origin, including monocytes, dendritic cells, and macrophages. These molecules appear to play a role in intracellular trafficking of molecules to lysosomes where antigen is processed for presentation. SEQ ID NO:35 is upregulated six to nine-fold, and 88% of its expression is found in dendritic cells, macrophages, and the promonocytic cell line THP-1. SEQ ID NO:35 encodes a transmembrane receptor (TREM-1) that is similar to the polymeric immunoglobulin receptor which binds and endocytoses immunoglobulins. Like that receptor, TREM-1 is expressed on neutrophils and monocytes, upregulated upon LPS stimulation, and induces secretion of chemokines and cytokines such as MCP-1,TNF-α, and IL-8. Because of these similarities, TREM-1 appears to act as a receptor for antibody bound to antigen. In addition, TREM-1 associates with a transmembrane protein DAP12, SEQ ID NO:64.

Molecules other than clathrin that are important in endocytosis and vesicle transport are upregulated in dendritic cells, such as cDNAs that encode myoferlin and VAT1, SEQ ID NOs:38 and 39, respectively. Myoferlin is upregulated approximately three-fold, and within the immune system 44% of sequences are found in monocytes, macrophages, granulocytes, and dendritic cells. Myoferlin regulates membrane fusion which is important in endocytosis of antigen. VAT1 is upregulated approximately four-fold, is expressed ubiquitously, and appears to be important in vesicular transport of amines.

Several proteases other than cathepsins are upregulated in dendritic cells. Several different types of metalloproteinase cDNAs are upregulated; for example, SEQ ID NOs:36, 40, and 45 are upregulated about 14-fold, three-fold, and three-fold, respectively. Metalloproteinases regulate processing of antigen into peptides for presentation on MHC class I molecules, cleave membrane bound proteins, such as TRANCE, and affect migration and differentiation of dendritic cells. Metalloproteinases are sometimes secreted extracellularly for antigen processing. DC secrete proteases which can process antigen extracellularly into peptides which bind to empty MHC class II molecules on the surface of the cell. Metalloproteinases and other proteases are upregulated during the initial innate immune response as well, wherein proteins on surfaces of pathogens are crosslinked and/or degraded. Other proteases upregulated include SEQ ID NOs:41, 43, and 46-49, which encompass aspartic, cysteine, and other peptidases. SEQ ID NOs:50 and 51 encode angiotensin-converting enzyme, which was upregulated three to six-fold. Dendritic cells also express cathepsin inhibitors in the vesicles in which MHC class II proteins are being formed. SEQ ID NO:44 encodes a cysteine proteinase that inhibits cathepsins L, S, and K, and was originally described as a marker of squamous cell carcinoma. In addition, protease inhibitors prevent host cell degradation by proteases produced by pathogens themselves. SEQ ID NOs:42 and 44 inhibit proteases and appear to function in this manner.

SEQ ID NOs:52-59 are cDNAs that encode growth factors upregulated in dendritic cells. These factors include those that have been described in other immune functions such as SEQ ID NO:52, which encodes an IL-1 receptor antagonist; SEQ ID NO:57, which encodes an IL-1 receptor homolog preferentially expressed on Th2 cells; and SEQ ID NO:58, which encodes the a chain of the IL-13 receptor. Others not previously associated with immune function are epididymal secretory protein, SEQ ID NOs:53-55 and bone-derived growth factor, SEQ ID NO:59. All of these cDNAs are upregulated from three to nine-fold. Although its cDNA encodes an epididymal-specific protein, SEQ ID NO:53 is a splice variant that contains a region that is preferentially expressed in hemic/ immune libraries, 67% of which are of monocytic, dendritic, macrophage, or granulocytic in origin.

SEQ ID NOs:60-78 are cDNAs that encode proteins that are upregulated in DC and play a role in signal transduction. SEQ ID NO:61 encodes Notch, which is upregulated about 10-fold and is a receptor involved in growth and differentiation in a wide variety of cells including T lymphocytes. Both kinases and phosphatases are upregulated in dendritic cells; for example, B lymphocyte serine/threonine protein kinase, SEQ ID NO:71, which transduces signals from CD40 and is upregulated 30-fold, and a G-protein coupled receptor (GRK5), SEQ ID NO:72, which is upregulated 20-fold. SEQ ID NO:77 encodes doc-1, an S-phase regulator and tumor suppressor in oral cancer that may induce apoptosis. A related sequence, SEQ ID NO:78 encodes doc-2, another tumor suppressor phosphoprotein that contains a potential transmembrane sequence at amino acids 493 to 513. SEQ ID NO:64 is upregulated about four-fold in dendritic cells, and in the immune system 40% of its expression is found in dendritic cells and granulocytes. SEQ ID NO:64 encodes DAP12, a receptor that has homology to the CD3 zeta chain of the T cell receptor complex and may trigger maturation and activation of dendritic cells through antigen uptake by TREM-1.

SEQ ID NOs:79-98 are cDNAs that are upregulated in DC and encode molecules involved in DNA transcription. In particular, about 40% of upregulated sequences involved in DNA transcription encode zinc finger proteins. SEQ ID NO:90 is upregulated about 30-fold and encodes a zinc finger homeobox protein. SEQ ID NO:94 is upregulated from about three to about six-fold and encodes a breast cancer tumor suppressor gene, bcsc-1, which has two potential transmembrane regions at amino acids 224 to 242 and 370 to 389. Bcsc-1 has three potential glycosylation sites, 7 potential casein kinase II phosphorylation sites, 7 potential protein kinase C phosphorylation sites, and 4 potential myristylation sites.

SEQ ID NOs:99-143 are sequences upregulated in DC that encode molecules involved in a variety of pathways. SEQ ID NOs:99-101 encode proteins involved in iron metabolism. SEQ ID NOs:102 and 103 encode transmembrane proteins involved in lipid metabolism. SEQ ID NO:104, which encodes apolipoprotein E, is upregulated five to 19-fold. SEQ ID NOs:105 and 106, which encode apolipoprotein C-I, are upregulated three to five-fold and 38% of its immune tissue expression is found in monocytes, dendritic cells, macrophages, and granulocytes. Apolipoprotein E is secreted by macrophages to dispose of cholesterol and other lipids. During the innate immune response, lipoprotein components from pathogens are among the first antigens encountered by dendritic cells and macrophages. Scavenger receptors take up these lipoproteins, which are then processed for antigen presentation. Byproducts such as cholesterol and other lipids are then expelled through transporters, such as the ABCA1 transporter expressed on macrophages. Other cDNAs that encode proteins that export molecules detrimental to the host cell and that are upregulated in dendritic cells include the multidrug resistance associated protein 3, SEQ ID NO:132, which is upregulated four to five-fold. In the immune system, SEQ ID NO:132 has an abundance of 45% in macrophages and LPS-activated monocytes. A number of other cDNAs encoding transporters are upregulated three to five-fold in dendritic cells; these include SEQ ID NOs:133-138, which encode anion transporters, and SEQ ID NOs:140 and 142, which encode chloride channels. Other molecules that protect dendritic cells during immune response are SEQ ID NOs:120-128, which encode the vacuolar H+ATPase subunits which are upregulated three to 12-fold in dendritic cells. Recent studies have shown that ATPase is upregulated during the differentiation of monocytes into macrophages and can serve to downregulate production of inflammatory cytokines such as TNF-α and IL-1 thus preventing deleterious effects of these cytokines on the host. SEQ ID NOs:129 and 130 encode glutathione-S-transferase homolog and glutathione-S-transferase, respectively, proteins which protect cells from oxidative stress and toxic foreign chemicals produced by pathogens. Upregulation of SEQ ID NOs:129 and 130 in DC may prevent damage from production of pro-inflammatory cytokines.

A number of enzymes and other cytosolic and lysosomal proteins involved in lipid metabolism are upregulated and preferentially expressed in libraries made from monocytes, dendritic cells, macrophages, and granulocytes. These molecules include SEQ ID NOs:107, which encodes a lipoprotein lipase upregulated four to six-fold; and SEQ ID NOs:113-115, which encode fatty acid binding protein and fatty acid binding protein homolog and are upregulated 10 to 30-fold. More than 40% of the immune system expression of SEQ ID NO:116, which encodes lysosomal acid lipase, occurs in monocytes, dendritic cells, macrophages, and granulocytes. SEQ ID NO:118, which encodes palmitoyl-protein thioesterase, is upregulated four to five fold and exhibits 17% abundance in immune cells, 40% of which are monocytes, dendritic cells, macrophages, and granulocytes. These molecules, which metabolize lipids of pathogenic origin, appear to help in the process of remodeling the dendritic plasma membrane and membranes of other lysosomal and other endocytic compartments.

SEQ ID NOs:144-151 are cDNAs that encode surface or secreted proteins that have a role in adhesion. SEQ ID NOs:145 and 146 encode osteopontin which is upregulated 11 to 29-fold and 56% of immune system expression occurs in activated DC, macrophages, and THP-1 cells. Osteopontin binds to the cell adhesion molecule CD44 and is induced by GM-CSF. Another molecule that may be involved in cell to cell contact is SEQ ID NO:144 which encodes kerato-epithelin and is upregulated about 10-fold. SEQ ID NO:147 is upregulated more than five-fold, expressed most abundantly in dendritic cells, macrophages, and T cells, and encodes a 14 kD lectin which chemoattracts monocytes and macrophages. SEQ ID NO:148 is upregulated 14.4-fold and encodes a member of the claudin protein family which plays a role in adhesion of cells through tight junctions.

SEQ ID NOs:152-165 are cDNAs upregulated in DC that encode molecules involved in cellular structure. As monocytes mature into DC, profound morphological changes occur that allow increased mobility and the formation of dendritic projections. These changes result in an increased surface area, thereby enhancing uptake of antigen. cDNAs, which are upregulated in dendritic cells and affect structure, encode α tubulin (SEQ ID NOs:152 and 157); vimentin (SEQ ID NO:153); gamma-actin (SEQ ID NOs:155 and 156); and beta-actin (SEQ ID NO:163). EB1 (SEQ ID NO:165) is upregulated almost four-fold and associates with microtubulues and adenomatous polyposis coli (APC) protein, which shuttles beta-catenin from the cytosol to the nucleus. APC is important in signaling through Wnt, a soluble protein that influences cell adhesion, growth, and differentiation.

SEQ ID NOs:166-176 are cDNAs upregulated in DC that encode molecules which regulate the innate immune response. SEQ ID NO:171 encodes Toll-like receptor 1 (TLR-1), a member of the IL-1 receptor superfamily thought to transduce signals that upregulate costimulatory molecules and cytokines. Immune responses to LPS and bacteria require certain Toll receptors, although the role of TLR-1 has not previously been identified. In Drosophila, the putative Toll receptor ligand is the product of a protease cascade. Other cDNAs that may participate in the innate immune response include SEQ ID NOs:172, 174, 175, and 176, which encode α-2-macroglobulin, factor XIIIa, transglutaminase, and thrombomodulin, respectively. α-2-macroglobulin binds proteases, inhibiting their activity and preventing damage from pathogenic proteases. Transglutaminase and factor XIIIa may participate in the crosslinking of proteins present on pathogens, enhancing phagocytosis. Thrombomodulin is a receptor for thrombin, which has both pro- and anticoagulant activities.

SEQ ID NOs:177-182 are cDNAs that are upregulated in DC and expressed preferentially in the immune system. SEQ ID NO:177, which encodes a transmembrane protein, DORA, is upregulated 14.5-fold, and 50% of its expression occurs in DC, macrophages, and granulocytes. SEQ ID NO:178, which encodes a membrane-associated protein, is upregulated almost five-fold, and 18% of its expression occurs in the immune system. SEQ ID NO:179, which encodes CD52, a glycosylated protein associated with the membrane, is upregulated from about three to about four-fold, and 35% of its expression occurs in the immune system. SEQ ID NOs:180 and 181, which encode variants of the tetraspanin CD53, are upregulated three to 20-fold, and 30% of their immune system expression occurs in macrophages, activated monocytes, dendritic cells, and granulocytes. SEQ ID NO:182 which encodes PIL protein is upregulated about four-fold; transcript analysis indicates that PIL protein is expressed in both dendritic cells and T cells.

SEQ ID NOs:183-200 are cDNAs that are upregulated in DC and encode transmembrane proteins. In particular, SEQ ID NOs:186 and 187 are upregulated about six-fold and about three-fold, respectively, and encode proteins that have signal peptides at the N-terminus. SEQ ID NO:189 is upregulated about four-fold and encodes a protein whose expression is preferentially found in dendritic cells and macrophages. SEQ ID NO:196, which is upregulated about four-fold, encodes a protein that has seven predicted transmembrane regions, and 44% of transcripts in the immune system are found in monocytes, macrophages, dendritic cells and granulocytes.

SEQ ID NOs:201-215 are cDNAs that are upregulated in DC and encode homologs of proteins with known function. SEQ ID NOs:201 and 202 encode variants of HU-K4 which has similarity to a phospholipase-D protein from Vaccinia virus. SEQ ID NO:202 is preferentially expressed in dendritic cells, and SEQ ID NO:201 is preferentially expressed in monocytes, dendritic cells, macrophages, and granulocytes. Several cDNAs encode homologs of proteins involved in immune response. SEQ ID NOs:203 and 204 encode a molecule that has approximately 35% identity to the IL-17 receptor, and 16% of their transcripts are found in the immune system. SEQ ID NO:205 encodes a protein similar to murine ClqC chain, a component of the complement system. SEQ ID NO:211 encodes a protein similar to a murine zinc finger protein and is upregulated 22.7-fold. Some 19% of SEQ ID NO:211 transcripts are found in hemic/immune tissues, preferentially macrophages. SEQ ID NO:212 encodes a novel GATA zinc finger transcription factor, with 65% identity to the murine GATA-5 cardiac transcription factor.

SEQ ID NOs:215-225 are cDNAs that are upregulated in DC and encode homologs of proteins of unknown function that are upregulated three to five-fold. SEQ ID NO:216 encodes a protein that contains seven potential transmembrane regions. SEQ ID NO:218 encodes a protein with eight potential transmembrane regions, and SEQ ID NO:220 encodes a protein with one predicted transmembrane region. SEQ ID NO:221 encodes a protein that has 12 potential transmembrane regions and is expressed preferentially in monocytes, dendritic cells, macrophages, and granulocytes. In addition, over 14% of transcripts of SEQ ID NOs:219 and 223-225 occurs in immune tissues. Over 30% of transcripts of SEQ ID NOs:219 and 221-225 in the immune system occurs in monocytes, dendritic cells, macrophages, and granulocytes.

SEQ ID NOs:226-241 are unique cDNAs upregulated in DC from three to 16-fold and contain potential open reading frames. Of these, SEQ ID NOs:228, 229, 233, 237, and 238 have a specific abundance ranging from 14 to 30% in hemic and immune tissues, while SEQ ID NOs:227 and 228 have a specific abundance in the immune system of 36% and 41%, respectively, in monocytes, dendritic cells, macrophages, and granulocytes. SEQ ID NOs:226, 230, and 239 are expressed 15.9-fold, 13.3-fold, and 12.7-fold, respectively, in DC over that of monocytes.

Table 4 lists cDNAs that are upregulated by mature DC. Survival and function of DC may be enhanced by factors such as SEQ ID NOs:242 and 243, which are upregulated three-fold and encode variants of IL-1 beta, a cytokine involved in regulating Th1 type immune responses; SEQ ID NO:249 which is upregulated between eight and 12-fold and encodes IL-8; SEQ ID NOs:250 and 251, which are upregulated from about three to about five-fold and encode variants of small inducible cytokine A4, a chemokine similar to macrophage inflammatory protein-1 beta. Survival may also be enhanced by upregulation by three-fold of SEQ ID NO:253, which encodes an inhibitor of apoptosis protein, MIHC. SEQ ID NOs:244 and 245 encode CD9, a tetraspanin which is upregulated from about three to about five-fold and interacts with the integrin family and other membrane proteins. Upregulation of CD9 may enhance DC cell migration and adhesion. SEQ ID NO:248 encodes I-TRAF, which interacts with TRAF, a necessary component for transducing signals from the TRANCE receptor. In addition, two cDNAs are unique, SEQ ID NO:256 and SEQ ID NO:257, which are upregulated about four-fold and three-fold, respectively.

Table 5 lists cDNAs that are downregulated by mature DC. SEQ ID NO:258 encodes neurogranin, which has no known immune function. Although preferentially expressed in the nervous system, neurogranin is expressed in a number of immune tissues, including dendritic cells, eosinophils, B cells, T cells, and the promonocyte line THP-1. SEQ ID NOs:259 and 260 encode two unique proteins, each downregulated about three-fold, which are similar to a zinc finger protein and ribonucleoprotein, respectively.

The cDNAs of the invention define a differential expression pattern against which to compare the expression pattern of biopsied and/or in vitro treated dendritic cells. Experimentally, differential expression of the cDNAs can be evaluated by methods including, but not limited to, differential display by spatial immobilization or by gel electrophoresis, genome mismatch scanning, representational discriminant analysis, clustering, transcript imaging and array technologies. These methods may be used alone or in combination.

The combination may be arranged on a substrate and hybridized with dendritic cells from subjects with diagnosed disorders to identify those sequences which are differentially expressed in dendritic cells from normal subjects versus dendritic cells from diseased subjects. This allows identification of those sequences of highest diagnostic and potential therapeutic value. In one embodiment, an additional set of cDNAs, such as cDNAs encoding signaling molecules, are arranged on the substrate with the combination. Such combinations may be useful in the elucidation of pathways which are affected in a particular disorder or to identify new, coexpressed, candidate, therapeutic molecules.

In another embodiment, the combination can be used for large scale genetic or gene expression analysis of a large number of novel, nucleic acid molecules. These samples are prepared by methods well known in the art and are from mammalian cells or tissues which are in a certain stage of development; have been treated with a known molecule or compound, such as a cytokine, growth factor, a drug, and the like; or have been extracted or biopsied from a mammal with a known or unknown condition, disorder, or disease before or after treatment. The sample nucleic acid molecules are hybridized to the combination for the purpose of defining a novel gene profile associated with that developmental stage, treatment, or disorder.

cDNAs and Their Uses

cDNAs can be prepared by a variety of synthetic or enzymatic methods well known in the art. cDNAs can be synthesized, in whole or in part, using chemical methods well known in the art (Caruthers et al. (1980) Nucleic Acids Symp Ser (7)215-233). Alternatively, cDNAs can be produced enzymatically or recombinantly, by in vitro or in vivo transcription.

Nucleotide analogs can be incorporated into cDNAs by methods well known in the art. The only requirement is that the incorporated analog must base pair with native purines or pyrimidines. For example, 2,6-diaminopurine can substitute for adenine and form stronger bonds with thymidine than those between adenine and thymidine. A weaker pair is formed when hypoxanthine is substituted for guanine and base pairs with cytosine. Additionally, cDNAs can include nucleotides that have been derivatized chemically or enzymatically.

cDNAs can be synthesized on a substrate. Synthesis on the surface of a substrate may be accomplished using a chemical coupling procedure and a piezoelectric printing apparatus as described by Baldeschweiler et al. (PCT publication WO95/25 1116). Alternatively, the cDNAs can be synthesized on a substrate surface using a self-addressable electronic device that controls when reagents are added as described by Heller et al. (U.S. Pat. No. 5,605,662). cDNAs can be synthesized directly on a substrate by sequentially dispensing reagents for their synthesis on the substrate surface or by dispensing preformed DNA fragments to the substrate surface. Typical dispensers include a micropipette delivering solution to the substrate with a robotic system to control the position of the micropipette with respect to the substrate. There can be a multiplicity of dispensers so that reagents can be delivered to the reaction regions efficiently.

cDNAs can be immobilized on a substrate by covalent means such as by chemical bonding procedures or UV irradiation. In one method, a cDNA is bound to a glass surface which has been modified to contain epoxide or aldehyde groups. In another method, a cDNA is placed on a polylysine coated surface and UV cross-linked to it as described by Shalon et al. (WO95/35505). In yet another method, a cDNA is actively transported from a solution to a given position on a substrate by electrical means (Heller, supra). cDNAs do not have to be directly bound to the substrate, but rather can be bound to the substrate through a linker group. The linker groups are typically about 6 to 50 atoms long to provide exposure of the attached cDNA. Preferred linker groups include ethylene glycol oligomers, diamines, diacids and the like. Reactive groups on the substrate surface react with a terminal group of the linker to bind the linker to the substrate. The other terminus of the linker is then bound to the cDNA. Alternatively, polynucleotides, plasmids or cells can be arranged on a filter. In the latter case, cells are lysed, proteins and cellular components degraded, and the DNA is coupled to the filter by UV cross-linking.

The cDNAs may be used for a variety of purposes. For example, the combination of the invention may be used on an array. The array, in turn, can be used in high-throughput methods for detecting a related polynucleotide in a sample, screening a plurality of molecules or compounds to identify a ligand, diagnosing autoimmune disease or cancer, following a treatment of cancer with in vitro manipulated dendritic cells, following treatment of autoimmune disease with agents that suppress dendritic cell function, or inhibiting or inactivating a therapeutically relevant gene related to the cDNA.

When the cDNAs of the invention are employed on a microarray, the cDNAs are arranged in an ordered fashion so that each cDNA is present at a specified location. Because the cDNAs are at specified locations on the substrate, the hybridization patterns and intensities, which together create a unique expression profile, can be interpreted in terms of expression levels of particular genes and can be correlated with a particular metabolic process, condition, disorder, disease, stage of disease, or treatment.

Hybridization

The cDNAs or fragments or complements thereof may be used in various hybridization technologies. The cDNAs may be labeled using a variety of reporter molecules by either PCR, recombinant, or enzymatic techniques. For example, a commercially available vector containing the cDNA is transcribed in the presence of an appropriate polymerase, such as T7 or SP6 polymerase, and at least one labeled nucleotide. Commercial kits are available for labeling and cleanup of such cDNAs. Radioactive (Amersham Pharmacia Biotech (APB), Piscataway N.J.), fluorescent (Operon Technologies, Alameda Calif.), and chemiluminescent labeling (Promega, Madison Wis.) are well known in the art.

A cDNA may represent the complete coding region of an mRNA or be designed or derived from unique regions of the mRNA or genomic molecule, an intron, a 3′ untranslated region, or from a conserved motif. The cDNA is at least 18 contiguous nucleotides in length and is usually single stranded. Such a cDNA may be used under hybridization conditions that allow binding only to an identical sequence, a naturally occurring molecule encoding the same protein, or an allelic variant. Discovery of related human and mammalian sequences may also be accomplished using a pool of degenerate cDNAs and appropriate hybridization conditions. Generally, a cDNA for use in Southern or northern hybridizations may be from about 400 to about 6000 nucleotides long. Such cDNAs have high binding specificity in solution-based or substrate-based hybridizations. An oligonucleotide, a fragment of the cDNA, may be used to detect a polynucleotide in a sample using PCR.

The stringency of hybridization is determined by G+C content of the cDNA, salt concentration, and temperature. In particular, stringency is increased by reducing the concentration of salt or raising the hybridization temperature. In solutions used for some membrane based hybridizations, addition of an organic solvent such as formamide allows the reaction to occur at a lower temperature. Hybridization may be performed with buffers, such as 5×saline sodium citrate (SSC) with 1% sodium dodecyl sulfate (SDS) at 60° C., that permit the formation of a hybridization complex between nucleic acid sequences that contain some mismatches. Subsequent washes are performed with buffers such as 0.2×SSC with 0.1% SDS at either 45° C. (medium stringency) or 65°-68° C. (high stringency). At high stringency, hybridization complexes will remain stable only where the nucleic acid molecules are completely complementary. In some membrane-based hybridizations, preferably 35% or most preferably 50%, formamide may be added to the hybridization solution to reduce the temperature at which hybridization is performed. Background signals may be reduced by the use of detergents such as Sarkosyl or Triton X-100 (Sigma Aldrich, St. Louis Mo.) and a blocking agent such as denatured salmon sperm DNA. Selection of components and conditions for hybridization are well known to those skilled in the art and are reviewed in Ausubel et al. (1997, Short Protocols in Molecular Biology, John Wiley & Sons, New York N.Y., Units 2.8-2.11, 3.18-3.19 and 4.6-4.9).

Dot-blot, slot-blot, low density and high density arrays are prepared and analyzed using methods known in the art. cDNAs from about 18 consecutive nucleotides to about 5000 consecutive nucleotides in length are contemplated by the invention and used in array technologies. The preferred number of cDNAs on an array is at least about 100,000, a more preferred number is at least about 40,000, an even more preferred number is at least about 10,000, and a most preferred number is at least about 600 to about 800. The array may be used to monitor the expression level of large numbers of genes simultaneously and to identify genetic variants, mutations, and SNPs. Such information may be used to determine gene function; to understand the genetic basis of a disorder; to diagnose a disorder; and to develop and monitor the activities of therapeutic agents being used to control or cure a disorder. (See, e.g., U.S. Pat. No. 5,474,796; WO95/11995; WO95/35505; U.S. Pat. No. 5,605,662; and U.S. Pat. No. 5,958,342.)

Screening and Purification Assays

A cDNA may be used to screen a library or a plurality of molecules or compounds for a ligand which specifically binds the cDNA. Ligands may be DNA molecules, RNA molecules, peptide nucleic acid molecules, peptides, proteins such as transcription factors, promoters, enhancers, repressors, and other proteins that regulate replication, transcription, or translation of the polynucleotide in the biological system. The assay involves combining the cDNA or a fragment thereof with the molecules or compounds under conditions that allow specific binding and detecting the bound cDNA to identify at least one ligand that specifically binds the cDNA.

In one embodiment, the cDNA may be incubated with a library of isolated and purified molecules or compounds and binding activity determined by methods such as a gel-retardation assay (U.S. Pat. No. 6,010,849) or a reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay. Protein binding may be confirmed by raising antibodies against the protein and adding the antibodies to the gel-retardation assay where specific binding will cause a supershift in the assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

The cDNA may be used to purify a ligand from a sample. A method for using a cDNA to purify a ligand would involve combining the cDNA or a fragment thereof with a sample under conditions to allow specific binding, recovering the bound cDNA, and using an appropriate agent to separate the cDNA from the purified ligand.

Protein Production and Uses

The full length cDNAs or fragment thereof may be used to produce purified proteins using recombinant DNA technologies described herein and taught in Ausubel et al. (supra; Units 16.1-16.62). One of the advantages of producing proteins by these procedures is the ability to obtain highly-enriched sources of the proteins thereby simplifying purification procedures.

The proteins may contain amino acid substitutions, deletions or insertions made on the basis of similarity in polarity, charge, solubility, hydrophobicity, hydrophilicity, and/or the amphipathic nature of the residues involved. Such substitutions may be conservative in nature when the substituted residue has structural or chemical properties similar to the original residue (e.g., replacement of leucine with isoleucine or valine) or they may be nonconservative when the replacement residue is radically different (e.g., a glycine replaced by a tryptophan). Computer programs included in LASERGENE software (DNASTAR, Madison Wis.), MACVECTOR software (Genetics Computer Group, Madison Wis.) and RasMol software (Roger Sayle, University of Massachusetts, Amherst Mass.) may be used to help determine which and how many amino acid residues in a particular portion of the protein may be substituted, inserted, or deleted without abolishing biological or immunological activity.

Expression of Encoded Proteins

Expression of a particular cDNA may be accomplished by cloning the cDNA into a vector and transforming this vector into a host cell. The cloning vector used for the construction of cDNA libraries in the LIFESEQ databases may also be used for expression. Such vectors usually contain a promoter and a polylinker useful for cloning, priming, and transcription. An exemplary vector may also contain the promoter for β-galactosidase, an amino-terminal methionine and the subsequent seven amino acid residues of β-galactosidase. The vector may be transformed into competent E. coli cells. Induction of the isolated bacterial strain with isopropylthiogalactoside (IPTG) using standard methods will produce a fusion protein that contains an N terminal methionine, the first seven residues of β-galactosidase, about residues of linker, and the protein encoded by the cDNA.

The cDNA may be shuttled into other vectors known to be useful for expression of protein in specific hosts. Oligonucleotides containing cloning sites and fragments of DNA sufficient to hybridize to stretches at both ends of the cDNA may be chemically synthesized by standard methods. These primers may then be used to amplify the desired fragments by PCR. The fragments may be digested with appropriate restriction enzymes under standard conditions and isolated using gel electrophoresis. Alternatively, similar fragments are produced by digestion of the cDNA with appropriate restriction enzymes and filled in with chemically synthesized oligonucleotides. Fragments of the coding sequence from more than one gene may be ligated together and expressed.

Signal sequences that dictate secretion of soluble proteins are particularly desirable as component parts of a recombinant sequence. For example, a chimeric protein may be expressed that includes one or more additional purification-facilitating domains. Such domains include, but are not limited to, metal-chelating domains that allow purification on immobilized metals, protein A domains that allow purification on immobilized immunoglobulin, and the domain utilized in the FLAGS extension/affinity purification system (Immunex, Seattle Wash.). The inclusion of a cleavable-linker sequence such as ENTEROKINASEMAX (Invitrogen, San Diego Calif.) between the protein and the purification domain may also be used to recover the protein.

Suitable host cells may include, but are not limited to, mammalian cells such as Chinese Hamster Ovary (CHO) and human 293 cells, insect cells such as Sf9 cells, plant cells such as Nicotiana tabacum, yeast cells such as Saccharomyces cerevisiae, and bacteria such as E. coli. For each of these cell systems, a useful vector may also include an origin of replication and one or two selectable markers to allow selection in bacteria as well as in a transformed eukaryotic host. Vectors for use in eukaryotic host cells may require the addition of 3′ poly(A) tail if the cDNA lacks poly(A).

Additionally, the vector may contain promoters or enhancers that increase gene expression. Many promoters are known and used in the art. Most promoters are host specific and exemplary promoters include SV40 promoters for CHO cells; T7 promoters for bacterial hosts; viral promoters and enhancers for plant cells; and PGH promoters for yeast. Adenoviral vectors with the rous sarcoma virus enhancer or retroviral vectors with long terminal repeat promoters may be used to drive protein expression in mammalian cell lines. Once homogeneous cultures of recombinant cells are obtained, large quantities of secreted soluble protein may be recovered from the conditioned medium and analyzed using chromatographic methods well known in the art. An alternative method for the production of large amounts of secreted protein involves the transformation of mammalian embryos and the recovery of the recombinant protein from milk produced by transgenic cows, goats, sheep, and the like.

In addition to recombinant production, proteins or portions thereof may be produced manually, using solid-phase techniques (Stewart et al. (1969) Solid-Phase Peptide Synthesis, W H Freeman, San Francisco Calif.; Merrifield (1963) J Am Chem Soc 5:2149-2154), or using machines such as the ABI 431A peptide synthesizer (Applied Biosystems, Foster City Calif.). Proteins produced by any of the above methods may be used as pharmaceutical compositions to treat disorders associated with null or inadequate expression of the genomic sequence.

Screening and Purification Assays

A protein or a portion thereof encoded by the cDNA may be used to screen a library or plurality of molecules or compounds for a ligand with specific binding affinity or to purify a molecule or compound from a sample. The protein or portion thereof employed in such screening may be free in solution, affixed to an abiotic or biotic substrate, or located intracellularly. For example, viable or fixed prokaryotic host cells that are stably transformed with recombinant nucleic acids that have expressed and positioned a protein on their cell surface can be used in screening assays. The cells are screened against a library or plurality of ligands and the specificity of binding or formation of complexes between the expressed protein and the ligand may be measured. The ligands may be DNA, RNA, or PNA molecules, agonists, antagonists, antibodies, immunoglobulins, inhibitors, peptides, pharmaceutical agents, proteins, drugs, or any other test molecule or compound that specifically binds the protein. An exemplary assay involves combining the mammalian protein or a portion thereof with the molecules or compounds under conditions that allow specific binding and detecting the bound protein to identify at least one ligand that specifically binds the protein.

This invention also contemplates the use of competitive drug screening assays in which neutralizing antibodies capable of binding the protein specifically compete with a test compound capable of binding to the protein, oligopeptide, or fragment thereof. One method for high throughput screening using very small assay volumes and very small amounts of test compound is described in U.S. Pat. No. 5,876,946. Molecules or compounds identified by screening may be used in a model system to evaluate their toxicity, diagnostic, or therapeutic potential.

Production of Antibodies

A protein encoded by a cDNA of the invention may be used to produce specific antibodies. Antibodies may be produced using an oligopeptide or a portion of the protein with inherent immunological activity. Methods for producing antibodies include: 1) injecting an animal, usually goats, rabbits, or mice, with the protein, or an antigenically-effective portion or an oligopeptide thereof, to induce an immune response; 2) engineering hybridomas to produce monoclonal antibodies; 3) inducing in vivo production in the lymphocyte population; or 4) screening libraries of recombinant immunoglobulins. Recombinant immunoglobulins may be produced as taught in U.S. Pat. No. 4,816,567.

Antibodies produced using the proteins of the invention are useful for the diagnosis of prepathologic disorders as well as the diagnosis of chronic or acute diseases characterized by abnormalities in the expression, amount, or distribution of the protein. A variety of protocols for competitive binding or immunoradiometric assays using either polyclonal or monoclonal antibodies specific for proteins are well known in the art. Immunoassays typically involve the formation of complexes between a protein and its specific binding molecule or compound and the measurement of complex formation. Immunoassays may employ a two-site, monoclonal-based assay that utilizes monoclonal antibodies reactive to two noninterfering epitopes on a specific protein or a competitive binding assay (Pound (1998) Immunochemical Protocols, Humana Press, Totowa N.J.).

Immunoassay procedures may be used to quantify expression of the protein in cell cultures, in subjects with a particular disorder or in model animal systems under various conditions. Increased or decreased production of proteins as monitored by immunoassay may contribute to knowledge of the cellular activities associated with developmental pathways, engineered conditions or diseases, or treatment efficacy. The quantity of a given protein in a given tissue may be determined by performing immunoassays on freeze-thawed detergent extracts of biological samples and comparing the slope of the binding curves to binding curves generated by purified protein.

Labeling of Molecules for Assay

A wide variety of reporter molecules and conjugation techniques are known by those skilled in the art and may be used in various cDNA, polynucleotide, protein, peptide or antibody assays. Synthesis of labeled molecules may be achieved using commercial kits for incorporation of a labeled nucleotide such as ³²P-dCTP, Cy3-dCTP or Cy5-dCTP or amino acid such as ³⁵S-methionine. Polynucleotides, cDNAs, proteins, or antibodies may be directly labeled with a reporter molecule by chemical conjugation to amines, thiols and other groups present in the molecules using reagents such as BIODIPY or FITC (Molecular Probes, Eugene Oreg.).

The proteins and antibodies may be labeled for purposes of assay by joining them, either covalently or noncovalently, with a reporter molecule that provides for a detectable signal. A wide variety of labels and conjugation techniques are known and have been reported in the scientific and patent literature including, but not limited to U.S. Pat. No. 3,817,837; 3,850,752; 3,939,350; 3,996,345; 4,277,437; 4,275,149; and 4,366,241.

Diagnostics

The cDNAs, or fragments thereof, may be used to detect and quantify differential gene expression; absence, presence, or excess expression of mRNAs; or to monitor mRNA levels during therapeutic intervention. Disorders associated with altered or differential expression include cancer, infectious disease, autoimmunity, allergy, and graft versus host disease. These cDNAs can also be utilized as markers of treatment efficacy against the disorders noted above and other disorders, conditions, and diseases over a period ranging from several days to months. The diagnostic assay may use hybridization or amplification technology to compare gene expression in a biological sample from a patient to standard samples in order to detect altered gene expression. Qualitative or quantitative methods for this comparison are well known in the art.

For example, the cDNA may be labeled by standard methods and added to a biological sample from a patient under conditions for hybridization complex formation. After an incubation period, the sample is washed and the amount of label (or signal) associated with hybridization complexes is quantified and compared with a standard value. If the amount of label in the patient sample is significantly altered in comparison to the standard value, then the presence of the associated condition, disease or disorder is indicated.

In order to provide a basis for the diagnosis of a condition, disease or disorder associated with gene expression, a normal or standard expression profile is established. This may be accomplished by combining a biological sample taken from normal subjects, either animal or human, with a probe under conditions for hybridization or amplification. Standard hybridization may be quantified by comparing the values obtained using normal subjects with values from an experiment in which a known amount of a substantially purified target sequence is used. Standard values obtained in this manner may be compared with values obtained from samples from patients who are symptomatic for a particular condition, disease, or disorder. Deviation from standard values toward those associated with a particular condition is used to diagnose that condition.

Such assays may also be used to evaluate the efficacy of a particular therapeutic treatment regimen in animal studies and in clinical trial or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to months.

Gene Expression Profiles

A gene expression profile comprises a plurality of cDNAs and a plurality of detectable hybridization complexes, wherein each complex is formed by hybridization of one or more probes to one or more complementary sequences in a sample. The cDNAs of the invention are used as elements on a microarray to analyze gene expression profiles. In one embodiment, the microarray is used to monitor the progression of disease. Researchers can assess and catalog the differences in gene expression between healthy and diseased tissues or cells. By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the microarray is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to characterize expression profiles associated with a particular condition, disorder or disease; or treatment of the condition, disorder or disease. Novel treatment regimens may be tested in these animal models using microarrays to establish and then follow expression profiles over time. In addition, microarrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

Assays Using Antibodies

Antibodies directed against epitopes on a protein encoded by a cDNA of the invention may be used in assays to quantify the amount of protein found in a particular human cell. Such assays include methods utilizing the antibody and a label to detect expression level under normal or disease conditions. The antibodies may be used with or without modification, and labeled by joining them, either covalently or noncovalently, with a labeling moiety.

Protocols for detecting and measuring protein expression using either polyclonal or monoclonal antibodies are well known in the art. Examples include ELISA, RIA, and fluorescent activated cell sorting (FACS). Such immunoassays typically involve the formation of complexes between the protein and its specific antibody and the measurement of such complexes. These and other assays are described in Pound (supra). The method may employ a two-site, monoclonal-based immunoassay utilizing monoclonal antibodies reactive to two non-interfering epitopes, or a competitive binding assay. (See, e.g., Coligan et al. (1997) Current Protocols in Immunology, Wiley-Interscience, New York N.Y.; Pound, supra)

Therapeutics

The cDNAs and fragments thereof can be used in gene therapy. cDNAs can be delivered ex vivo to target cells, such as cells of bone marrow. Once stable integration and transcription and or translation are confirmed, the bone marrow may be reintroduced into the subject. Expression of the protein encoded by the cDNA may correct a disorder associated with mutation of a normal sequence, reduction or loss of an endogenous target protein, or overepression of an endogenous or mutant protein. Alternatively, cDNAs may be delivered in vivo using vectors such as retrovirus, adenovirus, adeno-associated virus, herpes simplex virus, and bacterial plasmids. Non-viral methods of gene delivery include cationic liposomes, polylysine conjugates, artificial viral envelopes, and direct injection of DNA (Anderson (1998) Nature 392:25-30; Dachs et al. (1997) Oncol Res 9:313-325; Chu et al. (1998) J Mol Med 76(34): 184-192; Weiss et al. (1999) Cell Mol Life Sci 55(3):334-358; Agrawal (1996) Antisense Therapeutics, Humana Press, Totowa N.J.; and August et al. (1997) Gene Therapy (Advances in Pharmacology, Vol. 40), Academic Press, San Diego Calif.).

In addition, expression of a particular protein can be regulated through the specific binding of a fragment of a cDNA to a genomic sequence or an mRNA which encodes the protein or directs its transcription or translation. The cDNA can be modified or derivatized to any RNA-like or DNA-like material including peptide nucleic acids, branched nucleic acids, and the like. These sequences can be produced biologically by transforming an appropriate host cell with a vector containing the sequence of interest.

Molecules which regulate the activity of the cDNA or encoded protein are useful as therapeutics for cancer, infectious disease, autoimmunity, allergy, and graft versus host disease and for enhancing vaccines. Such molecules include agonists which increase the expression or activity of the polynucleotide or encoded protein, respectively; or antagonists which decrease expression or activity of the polynucleotide or encoded protein, respectively. In one aspect, an antibody which specifically binds the protein may be used directly as an antagonist or indirectly as a delivery mechanism for bringing a pharmaceutical agent to cells or tissues which express the protein.

Additionally, any of the proteins, or their ligands, or complementary nucleic acid sequences may be administered as pharmaceutical compositions or in combination with other appropriate therapeutic agents. Selection of the appropriate agents for use in combination therapy may be made by one of ordinary skill in the art, according to conventional pharmaceutical principles. The combination of therapeutic agents may act synergistically to affect the treatment or prevention of the conditions and disorders associated with an immune response. Using this approach, one may be able to achieve therapeutic efficacy with lower dosages of each agent, thus reducing the potential for adverse side effects. Further, the therapeutic agents may be combined with pharmaceutically-acceptable carriers including excipients and auxiliaries which facilitate processing of the active compounds into preparations which can be used pharmaceutically. Further details on techniques for formulation and administration used by doctors and pharmacists may be found in the latest edition of Remington's Pharmaceutical Sciences (Mack Publishing, Easton Pa.).

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of underexpression or overexpression of genes of interest and for the development of methods for diagnosis and treatment of diseases. A mammal inbred to overexpress a particular gene (for example, secreted in milk) may also serve as a convenient source of the protein expressed by that gene.

Transgenic Animal Models

Transgenic rodents that overexpress or underexpress a gene of interest may be inbred and used to model human diseases or to test therapeutic or toxic agents. (See, e.g., U.S. Pat. Nos. 5,175,383 and 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the potential to form embryonic tissues. When ES cells such as the mouse 129/SvJ cell line are placed in a blastocyst from the C57BL/6 mouse strain, they resume normal development and contribute to tissues of the live-born animal. ES cells are preferred for use in the creation of experimental knockout and knockin animals. The method for this process is well known in the art and the steps are: the cDNA is introduced into a vector, the vector is transformed into ES cells, transformed cells are identified and microinjected into mouse cell blastocysts, blastocysts are surgically transferred to pseudopregnant dams. The resulting chimeric progeny are genotyped and bred to produce heterozygous or homozygous strains.

Knockout Analysis

In gene knockout analysis, a region of a gene is enzymatically modified to include a non-natural intervening sequence such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene.

Knockin Analysis

ES cells can be used to create knockin humanized animals or transgenic animal models of human diseases. With knockin technology, a region of a human gene is injected into animal ES cells, and the human sequence integrates into the animal cell genome. Transgenic progeny or inbred lines are studied and treated with potential pharmaceutical agents to obtain information on the progression and treatment of the analogous human condition.

As described herein, the uses of the cDNAs, provided in the Sequence Listing of this application, and their encoded proteins are exemplary of known techniques and are not intended to reflect any limitation on their use in any technique that would be known to the person of average skill in the art. Furthermore, the cDNAs provided in this application may be used in molecular biology techniques that have not yet been developed, provided the new techniques rely on properties of nucleotide sequences that are currently known to the person of ordinary skill in the art, e.g., the triplet genetic code, specific base pair interactions, and the like. Likewise, reference to a method may include combining more than one method for obtaining or assembling full length cDNA sequences that will be known to those skilled in the art. It is also to be understood that this invention is not limited to the particular methodology, protocols, and reagents described, as these may vary. It is also understood that the terminology used herein is for the purpose of describing particular embodiments only, and is not intended to limit the scope of the present invention which will be limited only by the appended claims. The examples below are provided to illustrate the subject invention and are not included for the purpose of limiting the invention.

### EXAMPLES

### I Construction of cDNA Libraries

RNA was purchased from Clontech Laboratories (Palo Alto Calif.) or isolated from various tissues. Some tissues were homogenized and lysed in guanidinium isothiocyanate, while others were homogenized and lysed in phenol or in a suitable mixture of denaturants, such as TRIZOL reagent (Life Technologies, Rockville Md.). The resulting lysates were centrifuged over CsCl cushions or extracted with chloroform. RNA was precipitated with either isopropanol or ethanol and sodium acetate, or by other routine methods.

Phenol extraction and precipitation of RNA were repeated as necessary to increase RNA purity. In most cases, RNA was treated with DNAse. For most libraries, poly(A) RNA was isolated using oligo d(T)-coupled paramagnetic particles (Promega), OLIGOTEX latex particles (Qiagen, Valencia Calif.), or an OLIGOTEX mRNA purification kit (Qiagen). Alternatively, poly(A) RNA was isolated directly from tissue lysates using other kits, including the POLY(A)PURE mRNA purification kit (Ambion, Austin Tex.).

In some cases, Stratagene (La Jolla Calif.) was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies) using the recommended procedures or similar methods known in the art. (See Ausubel, supra, Units 5.1 through 6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (APB) or preparative agarose gel electrophoresis. cDNAs were ligated into compatible restriction enzyme sites of the polylinker of the pBLUESCRIPT phagemid (Stratagene), pSPORT1 plasmid (Life Technologies), or pinch plasmid (Incite Genomic, Palo Alto Calif.). Recombinant plasmids were transformed into XL1-BLUE, XL1-BLUEMRF, or SOLR competent E. coli cells (Stratagene) or DH5a, DH10B, or ELECTROMAX DH10B competent E. coli cells (Life Technologies).

In some cases, libraries were superinfected with a 5×excess of the helper phage, M13K07, according to the method of Vieira et al. (1987, Methods Enzymol 153:3-11) and normalized or subtracted using a methodology adapted from Soares (1994, Proc Natl Acad Sci 91:9228-9232), Swaroop et al. (1991, Nucl Acids Res 19:1954), and Bonaldo et al. (1996, Genome Res 6:791-806). The modified Soares normalization procedure was utilized to reduce the repetitive cloning of highly expressed high abundance cDNAs while maintaining the overall sequence complexity of the library. Modification included significantly longer hybridization times which allowed for increased gene discovery rates by biasing the normalized libraries toward those infrequently expressed low-abundance cDNAs which are poorly represented in a standard transcript image (Soares et al., supra).

II Isolation and Sequencing of cDNA Clones

Plasmids were recovered from host cells by in vivo excision using the UNIZAP vector system (Stratagene) or by cell lysis. Plasmids were purified using one of the following: the Magic or WIZARD MINIPREPS DNA purification system (Promega); the AGTC MINIPREP purification kit (Edge BioSystems, Gaithersburg Md.); the QIAWELL 8, QIAWELL 8 Plus, or QIAWELL 8 Ultra plasmid purification systems, or the REAL PREP 96 plasmid purification kit (QIAGEN). Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao (1994) Anal Biochem 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes) and a FLUOROSKAN II fluorescence scanner (Labsystems Oy, Helsinki, Finland).

cDNA sequencing reactions were processed using standard methods or high-throughput instrumentation such as the ABI CATALYST 800 thermal cycler (Applied Biosystems) or the DNA ENGINE thermal cycler (MJ Research, Watertown Mass.) in conjunction with the HYDRA microdispenser (Robbins Scientific, Sunnyvale Calif.) or the MICROLAB 2200 system (Hamilton, Reno Nev.). cDNA sequencing reactions were prepared using reagents provided by APB or supplied in ABI sequencing kits such as the ABI PRISM BIGDYE cycle sequencing kit (Applied Biosystems). Electrophoretic separation of cDNA sequencing reactions and detection of labeled cDNAs were carried out using the MEGABACE 1000 DNA sequencing system (APB); the ABI PRISM 373 or 377 sequencing systems (Applied Biosystems) in conjunction with standard ABI protocols and base calling software; or other sequence analysis systems known in the art. Reading frames within the cDNA sequences were identified using standard methods (reviewed in Ausubel, supra, Unit 7.7).

III Extension of cDNA Sequences

Nucleic acid sequences were extended using the cDNA clones and oligonucleotide primers. One primer was synthesized to initiate 5′ extension of the known fragment, and the other, to initiate 3′ extension of the known fragment. The initial primers were designed using OLIGO primer analysis software (Molecular Biology Insights, Cascade Colo.), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed. Preferred libraries are ones that have been size-selected to include larger cDNAs. Also, random primed libraries are preferred because they will contain more sequences with the 5′ and upstream regions of genes. A randomly primed library is particularly useful if an oligo d(T) library does not yield a full-length cDNA.

High fidelity amplification was obtained by PCR using methods well known in the art. PCR was performed in 96-well plates using the DNA ENGINE thermal cycler (MJ Research). The reaction mix contained DNA template, 200 nmol of each primer, reaction buffer containing Mg²⁺, (NH₄)₂SO₄, and β-mercaptoethanol, Taq DNA polymerase (APB), ELONGASE enzyme (Life Technologies), and Pfu DNA polymerase (Stratagene), with the following parameters for primer pair PCI A and PCI B (Incite Genomic): Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ (Stratagene) were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN reagent (0.25% reagent in 1×TE, v/v; Molecular Probes) and 0.5 μl of undiluted PCR product into each well of an opaque fluorimeter plate (Corning Costar, Acton Mass.) and allowing the DNA to bind to the reagent. The plate was scanned in a FLUOROSKAN II (Labsystems Oy) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleic acids were desalted and concentrated, transferred to 384-well plates, digested with CviJI cholera virus endonuclease (Molecular Biology Research, Madison Wis.), and sonicated or sheared prior to religation into pUC18 vector (APB). For shotgun sequencing, the digested nucleic acids were separated on low concentration (0.6 to 0.8%) agarose gels, fragments were excised, and agar digested with AGARACE enzyme (Promega). Extended clones were religated using T4 DNA ligase (New England Biolabs, Beverly Mass.) into pUC18 vector (APB), treated with Pfu DNA polymerase (Stratagene) to fill-in restriction site overhangs, and transformed into competent E. coli cells. Transformed cells were selected on antibiotic-containing media, and individual colonies were picked and cultured overnight at 37° C. in 384-well plates in LB/2×carbenicillin liquid media.

The cells were lysed, and DNA was amplified by PCR using Taq DNA polymerase (APB) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified using PICOGREEN reagent (Molecular Probes) as described above. Samples with low DNA recoveries were reamplified using the same conditions described above. Samples were diluted with 20% dimethylsulfoxide (DMSO; 1:2, v/v), and sequenced using DYENAMIC energy transfer sequencing primers and the DYENAMIC DIRECT cycle sequencing kit (APB) or the ABI PRISM BIGDYE terminator cycle sequencing kit (Applied Biosystems).

IV Assembly and Analysis of Sequences

Component nucleotide sequences from chromatograms were subjected to PHRED analysis (Phil Green, University of Washington, Seattle Wash.) and assigned a quality score. The sequences having at least a required quality score were subject to various pre-processing algorithms to eliminate low quality 3′ ends, vector and linker sequences, polyA tails, Alu repeats, mitochondrial and ribosomal sequences, bacterial contamination sequences, and sequences smaller than 50 base pairs. Sequences were screened using the BLOCK 2 program (Incite Genomic), a motif analysis program based on sequence information contained in the SWISS-PROT and PROSITE databases (Bairoch et al. (1997) Nucleic Acids Res 25:217-221; Attwood et al. (1997) J Chem Inf Comput Sci 37:417424).

Processed sequences were subjected to assembly procedures in which the sequences were assigned to bins, one sequence per bin. Sequences in each bin were assembled to produce consensus sequences, templates. Subsequent new sequences were added to existing bins using BLAST (Altschul (supra); Altschul et al. (supra); Karlin et al. (1988) Proc Natl Acad Sci 85:841-845), BLASTn (vers.1.4, WashU), and CROSSMATCH software (Phil Green, supra). Candidate pairs were identified as all BLAST hits having a quality score greater than or equal to 150. Alignments of at least 82% local identity were accepted into the bin. The component sequences from each bin were assembled using PHRAP (Phil Green, supra). Bins with several overlapping component sequences were assembled using DEEP PHRAP (Phil Green, supra).

Bins were compared against each other, and those having local similarity of at least 82% were combined and reassembled. Reassembled bins having templates of insufficient overlap (less than 95% local identity) were re-split. Assembled templates were also subjected to analysis by STITCHER/EXON MAPPER algorithms which analyzed the probabilities of the presence of splice variants, alternatively spliced exons, splice junctions, differential expression of alternative spliced genes across tissue types, disease states, and the like. These resulting bins were subjected to several rounds of the above assembly procedures to generate the template sequences found in the LIFESEQ GOLD database (Incite Genomic).

The assembled templates were annotated using the following procedure. Template sequences were analyzed using BLASTn (vers. 2.0, NCBI) versus GBpri (GenBank vers. 116). “Hits” are defined as an exact match having from 95% local identity over 200 base pairs through 100% local identity over 100 base pairs, or a homolog match having an E-value equal to or greater than 1×10⁻⁸. (The “E-value” quantifies the statistical probability that a match between two sequences occurred by chance). The hits were subjected to frameshift FASTx versus GENPEPT (GenBank version 109). In this analysis, a homolog match was defined as having an E-value of 1×10⁻⁸. The assembly method used above was described in U.S. Ser. No. 09/276,534, filed Mar. 25, 1999, and the LIFESEQ GOLD user manual (Incite Genomic).

Following assembly, template sequences were subjected to motif, BLAST, Hidden Markov Model (HMM; Pearson and Lipman (1988) Proc Natl Acad Sci 85:2444-2448; Smith and Waterman (1981) J Mol Biol 147:195-197), and functional analyses, and categorized in protein hierarchies using methods described in U.S. Ser. No. 08/812,290, filed Mar. 6, 1997; U.S. Ser. No. 08/947,845, filed Oct. 9, 1997; U.S. Pat. No. 5,953,727; and U.S. Ser. No. 09/034,807, filed Mar. 4, 1998. Template sequences may be further queried against public databases such as the GenBank rodent, mammalian, vertebrate, eukaryote, prokaryote, and human EST databases.

V Selection of Sequences, Microarray Preparation and Use

Incite clones represent template sequences derived from the LIFESEQ GOLD assembled human sequence database (Incite Genomic). In cases where more than one clone was available for a particular template, the 5′-most clone in the template was used on the microarray. The GENEALBUM GEM series 1-6 microarrays (Incite Genomic) contain 52,616 array elements which represent 17,472 annotated clusters and 35,144 unannotated clusters. The LIFEGEM 1.0 microarray (Incite Genomic) contains 9,203 array elements which represent 4,791 annotated genes and 1,861 unannotated clusters. For the UNIGEM series microarrays (Incite Genomic), Incite clones were mapped to non-redundant Unigene clusters (Unigene database (build 46), NCBI; Shuler (1997) J Mol Med 75:694-698), and the 5′ clone with the strongest BLAST alignment (at least 90% identity and 100 bp overlap) was chosen, verified, and used in the construction of the microarray. The UNIGEM V microarray (Incite Genomic) contains 9,182 array elements which represent 5,815 annotated genes and 2,699 unannotated clusters. UNIGEM V 2.0 microarray (Incite Genomic) contains 7075 array elements which represent 4610 annotated genes and 2,184 unannotated clusters. The HUMAN GENOME GEM series 1 microarray (Incite Genomic) contains 9,766 array elements which represent 7,612 annotated clusters and 1,382 unannotated clusters. Tables 2-5 show the GenBank annotations for SEQ ID NOs:1-260 of this invention as produced by BLAST analysis. LIFEGEM 1.0, GENEALBUM 1-6, and UNIGEM V microarrays were used to compare cDNA expression between monocytes and immature DC. LIFEGEM 1.0, UNIGEM V 2.0, and HUMAN GENOME GEM 1 were used to compare cDNA expression between mature DC and TRANCE-treated mature DC.

To construct microarrays, cDNAs were amplified from bacterial cells using primers complementary to vector sequences flanking the cDNA insert. Thirty cycles of PCR increased the initial quantity of cDNAs from 1-2 ng to a final quantity greater than 5 μg. Amplified cDNAs were then purified using SEPHACRYL-400 columns (APB). Purified cDNAs were immobilized on polymer-coated glass slides. Glass microscope slides (Corning, Corning N.Y.) were cleaned by ultrasound in 0.1% SDS and acetone, with extensive distilled water washes between and after treatments. Glass slides were etched in 4% hydrofluoric acid (VWR Scientific Products, West Chester Pa.), washed thoroughly in distilled water, and coated with 0.05% aminopropyl silane (Sigma Aldrich) in 95% ethanol. Coated slides were cured in a 110° C. oven. cDNAs were applied to the coated glass substrate using a procedure described in U.S. Pat. No. 5,807,522. One microliter of the cDNA at an average concentration of 100 ng/ul was loaded into the open capillary printing element by a high-speed robotic apparatus which then deposited about 5 nl of cDNA per slide.

Microarrays were UV-crosslinked using a STRATALINKER UV-crosslinker (Stratagene), and then washed at room temperature once in 0.2% SDS and three times in distilled water. Non-specific binding sites were blocked by incubation of microarrays in 0.2% casein in phosphate buffered saline (Tropix, Bedford Mass.) for 30 minutes at 60° C. followed by washes in 0.2% SDS and distilled water as before.

VI Preparation of Samples

Preparation of Monocytes and Immature Dendritic Cells

Peripheral blood mononuclear cells (PBMCs) were isolated from freshly obtained peripheral blood of four healthy donors by centrifugation of the lymphocyte enriched blood fraction over a HYPAQUE ficoll gradient (Sigma-Aldrich). The PBMCs were allowed to adhere to plastic in Iscove's Modified Dulbecco's Medium supplemented with 10% fetal bovine serum, 200 nM glutamine, and 200 nM each penicillin and streptomycin for 4 hours to separate monocytes from other nonadherent cells. One-half of the monocytes was cultured for seven days in 10 ng/ml GM-CSF (Peprotech, Rocky Hill N.J.), and the other half was cultured for seven days with 10 ng/ml GM-CSF and 10 ng/ml IL-4 (Peprotech) to produce immature dendritic cells.

Preparation of Mature Dendritic Cells

Monocytes were isolated as described above and were incubated with 10 ng/ml GM-CSF and 10 ng/ml IL-4 for 13 days to generate immature DC. The DC were activated with anti-CD40 (Biodesign International, Kennebunk Me.) at 10 ng/ml for 24 hours. The DC were divided into half, and each half further divided into thirds. Each third was cultured with soluble human TRANCE protein (Peprotech Inc.) at 10 ng/ml for 2, 8, and 24 hours, respectively, or cultured without TRANCE for 2, 8, and 24 hours, respectively.

Isolation and Labeling of Sample cDNAs

Cells were harvested and lysed in 1 ml of TRIZOL reagent (5×10⁶ cells/ml; Life Technologies). The lysates were vortexed thoroughly, incubated at room temperature for 2-3 minutes, and extracted with 0.5 ml chloroform. The extract was mixed, incubated at room temperature for 5 minutes, and centrifuged at 16,000 g for 15 minutes at 4° C. The aqueous layer was collected and an equal volume of isopropanol was added. Samples were mixed, incubated at room temperature for 10 minutes, and centrifuged at 16,000 g for 20 minutes at 4° C. The supernatant was removed and the RNA pellet was washed with 1 ml of 70% ethanol, centrifuged at 16,000 g at 4° C., and resuspended in RNAse-free water. The concentration of the RNA was determined by measuring the optical density at 260 nm.

Poly(A) RNA was prepared using an OLIGOTEX mRNA kit (QIAGEN) with the following modifications: OLIGOTEX beads were washed in tubes instead of on spin columns, resuspended in elution buffer, and then loaded onto spin columns to recover mRNA. To obtain maximum yield, the mRNA was eluted twice.

Each poly(A) RNA sample was reverse transcribed using MMLV reverse-transcriptase, 0.05 pg/μl oligo-d(T) primer (21mer), 1×first strand buffer, 0.03 units/ul RNase inhibitor, 500 uM dATP, 500 uM dGTP, 500 uM dTTP, 40 uM dCTP, and 40 uM either dCTP-Cy3 or dCTP-Cy5 (APB). The reverse transcription reaction was performed in a 25 ml volume containing 200 ng poly(A) RNA using the GEMBRIGHT kit (Incite Genomic). Specific control poly(A) RNAs (YCFRO6, YCFR45, YCFR67, YCFR85, YCFR43, YCFR22, YCFR23, YCFR25, YCFR44, YCFR26) were synthesized by in vitro transcription from non-coding yeast genomic DNA (W. Lei, unpublished). As quantitative controls, control mRNAs (YCFRO6, YCFR45, YCFR67, and YCFR85) at 0.002 ng, 0.02 ng, 0.2 ng, and 2 ng were diluted into reverse transcription reaction at ratios of 1:100,000, 1:10,000, 1:1000, 1:100 (w/w) to sample mRNA, respectively. To sample differential expression patterns, control mRNAs (YCFR43, YCFR22, YCFR23, YCFR25, YCFR44, YCFR26) were diluted into reverse transcription reaction at ratios of 1:3, 3:1, 1:10, 10:1, 1:25, 25:1 (w/w) to sample mRNA. Reactions were incubated at 37° C. for 2 hr, treated with 2.5 ml of 0.5M sodium hydroxide, and incubated for 20 minutes at 85° C. to the stop the reaction and degrade the RNA.

cDNAs were purified using two successive CHROMA SPIN 30 gel filtration spin columns (Clontech). Cy3- and Cy5-labeled reaction samples were combined as described below and ethanol precipitated using 1 ml of glycogen (1 mg/ml), 60 ml sodium acetate, and 300 ml of 100% ethanol. The cDNAs were then dried to completion using a SpeedVAC system (Savant Instruments, Holbrook N.Y.) and resuspended in 14 μl 5×SSC/0.2% SDS.

VII Hybridization and Detection

Hybridization reactions contained 9 μl of sample mixture containing 0.2 μg each of Cy3 and CyS labeled cDNA synthesis products in 5×SSC, 0.2% SDS hybridization buffer. The mixture was heated to 65° C. for 5 minutes and was aliquoted onto the microarray surface and covered with an 1.8 cm² coverslip. The microarrays were transferred to a waterproof chamber having a cavity just slightly larger than a microscope slide. The chamber was kept at 100% humidity internally by the addition of 140 μl of 5×SSC in a corner of the chamber. The chamber containing the microarrays was incubated for about 6.5 hours at 60° C. The microarrays were washed for 10 min at 45° C. in low stringency wash buffer (1×SSC, 0.1% SDS), three times for 10 minutes each at 45° C. in high stringency wash buffer (0.1×SSC), and dried.

Reporter-labeled hybridization complexes were detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light was focused on the microarray using a 20× microscope objective (Nikon, Melville N.Y.). The slide containing the microarray was placed on a computer-controlled X-Y stage on the microscope and raster-scanned past the objective. The 1.8 cm×1.8 cm microarray used in the present example was scanned with a resolution of 20 micrometers.

In two separate scans, the mixed gas multiline laser excited the two fluorophores sequentially. Emitted light was split, based on wavelength, into two photomultiplier tube detectors (PMT R1477; Hamamatsu Photonics Systems, Bridgewater N.J.) corresponding to the two fluorophores. Appropriate filters positioned between the microarray and the photomultiplier tubes were used to filter the signals. The emission maxima of the fluorophores used were 565 nm for Cy3 and 650 nm for CyS. Each microarray was typically scanned twice, one scan per fluorophore using the appropriate filters at the laser source, although the apparatus was capable of recording the spectra from both fluorophores simultaneously.

The sensitivity of the scans was calibrated using the signal intensity generated by a cDNA control species. Samples of the calibrating cDNA were separately labeled with the two fluorophores and identical amounts of each were added to the hybridization mixture. A specific location on the microarray contained a complementary DNA sequence, allowing the intensity of the signal at that location to be correlated with a weight ratio of hybridizing species of 1:100,000.

The output of the photomultiplier tube was digitized using a 12-bit RTI-835H analog-to-digital (A/D) conversion board (Analog Devices, Norwood, Mass.) installed in an IBM-compatible PC computer. The digitized data were displayed as an image where the signal intensity was mapped using a linear 20-color transformation to a pseudocolor scale ranging from blue (low signal) to red (high signal). The data was also analyzed quantitatively. Where two different fluorophores were excited and measured simultaneously, the data were first corrected for optical crosstalk (due to overlapping emission spectra) between the fluorophores using each fluorophore's emission spectrum.

A grid was superimposed over the fluorescence signal image such that the signal from each spot was centered in each element of the grid. The fluorescence signal within each element was then integrated to obtain a numerical value corresponding to the average intensity of the signal. The software used for signal analysis was the GEMTOOLS gene expression analysis program (Incite Genomic). Significance was defined as signal to background ratio exceeding 2×and area hybridization exceeding 40%.

VIII Data Analysis and Results

Array elements that exhibited at least a 2.5-fold change in expression at one or more time points, a signal intensity over 250 units, a signal-to-background ratio of at least 2.5, and an element spot size of at least 40% were identified as differentially expressed using the GEMTOOLS program (Incite Genomic). Differential expression is reported as the balanced differential expression value for each clone representing a gene on the microarray. The cDNAs that are differentially expressed are shown in Tables 2-5. Table 2 identifies downregulated cDNAs in immature DC. Table 3 identifies upregulated cDNAs in immature DC. Tables 4 and 5 identify cDNAs upregulated or downregulated, respectively, in mature DC. The cDNAs are identified by their SEQ ID NO and TEMPLATE ID, and by the description associated with at least a fragment of a polynucleotide found in GenBank. The descriptions were obtained using the sequences of the Sequence Listing and BLAST analysis.

IX Other Hybridization Technologies and Analyses

Other hybridization technologies utilize a variety of substrates such as nylon membranes, capillary tubes, etc. Arranging cDNAs on polymer coated slides is described in Example V; sample cDNA preparation and hybridization and analysis using polymer coated slides is described in examples VI and VII, respectively.

The cDNAs are applied to a membrane substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37° C. for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above.

Hybridization probes derived from cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100° C. for five min and briefly centrifuging. The denatured cDNA is then added to a REDIPRIME tube (APB), gently mixed until blue color is evenly distributed, and briefly centrifuged. Five microliters of [³²P]dCTP is added to the tube, and the contents are incubated at 37° C. for 10 min. The labeling reaction is stopped by adding 5 μl of 0.2M EDTA, and probe is purified from unincorporated nucleotides using a PROBEQUANT G-50 microcolumn (APB). The purified probe is heated to 100° C. for five min and then snap cooled for two min on ice.

Membranes are pre-hybridized in hybridization solution containing 1% Sarkosyl and 1×high phosphate buffer (0.5 M NaCl, 0.1 M Na₂HPO₄, 5 mM EDTA, pH 7) at 55° C. for two hr. The probe, diluted in 15 ml fresh hybridization solution, is then added to the membrane. The membrane is hybridized with the probe at 55° C. for 16 hr. Following hybridization, the membrane is washed for 15 min at 25° C. in 1 mM Tris (pH 8.0), 1% Sarkosyl, and four times for 15 min each at 25° C. in 1 mM Tris (pH 8.0). To detect hybridization complexes, XOMAT-AR film (Eastman Kodak, Rochester N.Y.) is exposed to the membrane overnight at −70° C., developed, and examined.

X Further Characterization of Differentially Expressed cDNAs and Proteins

Clones were blasted against the LIFESEQ Gold 5.1 database (Incite Genomic) and an Incite template and its sequence variants were chosen for each clone. The template and variant sequences were blasted against GenBank database to acquire annotation. The nucleotide sequences were translated into amino acid sequence which was blasted against the GenPept and other protein databases to acquire annotation and characterization, i.e., structural motifs.

Percent sequence identity can be determined electronically for two or more amino acid or nucleic acid sequences using the MEGALIGN program (DNASTAR). The percent identity between two amino acid sequences is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage identity.

Sequences with conserved protein motifs may be searched using the BLOCKS search program. This program analyses sequence information contained in the Swiss-Prot and PROSITE databases and is useful for determining the classification of uncharacterized proteins translated from genomic or cDNA sequences (Bairoch et al.(supra); Attwood et al. (supra). PROSITE database is a useful source for identifying functional or structural domains that are not detected using motifs due to extreme sequence divergence. Using weight matrices, these domains are calibrated against the SWISS-PROT database to obtain a measure of the chance distribution of the matches.

The PRINTS database can be searched using the BLIMPS search program to obtain protein family “fingerprints”. The PRINTS database complements the PROSITE database by exploiting groups of conserved motifs within sequence alignments to build characteristic signatures of different protein families. For both BLOCKS and PRINTS analyses, the cutoff scores for local similarity were: >1300=strong, 1000-1300=suggestive; for global similarity were: p<exp-3; and for strength (degree of correlation) were: >1300=strong, 1000-1300=weak.

XI Expression of the Encoded Protein

Expression and purification of a protein encoded by a cDNA of the invention is achieved using bacterial or virus-based expression systems. For expression in bacteria, cDNA is subcloned into a vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into bacterial hosts, such as BL21(DE3). Antibiotic resistant bacteria express the protein upon induction with IPTG. Expression in eukaryotic cells is achieved by infecting Spodoptera frugiperda (Sf9) insect cells with recombinant baculovirus, Autographica californica nuclear polyhedrosis virus. The polyhedrin gene of baculovirus is replaced with the cDNA by either homologous recombination or bacterial-mediated transposition involving transfer plasmid intermediates. Viral infectivity is maintained and the strong polyhedrin promoter drives high levels of transcription.

For ease of purification, the protein is synthesized as a fusion protein with glutathione-S-transferase (GST; APB) or a similar alternative such as FLAG. The fusion protein is purified on immobilized glutathione under conditions that maintain protein activity and antigenicity. After purification, the GST moiety is proteolytically cleaved from the protein with thrombin. A fusion protein with FLAG, an 8-amino acid peptide, is purified using commercially available monoclonal and polyclonal anti-FLAG antibodies (Eastman Kodak, Rochester N.Y.).

XII Production of Specific Antibodies

A denatured protein from a reverse phase HPLC separation is obtained in quantities up to 75 mg. This denatured protein is used to immunize mice or rabbits following standard protocols. About 100 μg is used to immunize a mouse, while up to 1 mg is used to immunize a rabbit. The denatured protein is radioiodinated and incubated with murine B-cell hybridomas to screen for monoclonal antibodies. About 20 mg of protein is sufficient for labeling and screening several thousand clones.

In another approach, the amino acid sequence translated from a cDNA of the invention is analyzed using PROTEAN software (DNASTAR) to determine regions of high antigenicity, essentially antigenically-effective epitopes of the protein. The optimal sequences for immunization are usually at the C-terminus, the N-terminus, and those intervening, hydrophilic regions of the protein that are likely to be exposed to the external environment when the protein is in its natural conformation. Typically, oligopeptides about 15 residues in length are synthesized using an ABI 431 peptide synthesizer (Applied Biosystems) using Fmoc-chemistry and then coupled to keyhole limpet hemocyanin (KLH; Sigma Aldrich) by reaction with M-maleimidobenzoyl-N-hydroxysuccinimide ester. If necessary, a cysteine may be introduced at the N-terminus of the peptide to permit coupling to KLH. Rabbits are immunized with the oligopeptide-KLH complex in complete Freund's adjuvant. The resulting antisera are tested for antipeptide activity by binding the peptide to plastic, blocking with 1% BSA, reacting with rabbit antisera, washing, and reacting with radioiodinated goat anti-rabbit IgG.

Hybridomas are prepared and screened using standard techniques. Hybridomas of interest are detected by screening with radioiodinated protein to identify those fusions producing a monoclonal antibody specific for the protein. In a typical protocol, wells of 96 well plates (FAST, Becton-Dickinson, Palo Alto Calif.) are coated with affinity-purified, specific rabbit-anti-mouse (or suitable anti-species Ig) antibodies at 10 mg/ml. The coated wells are blocked with 1% BSA and washed and exposed to supernatants from hybridomas. After incubation, the wells are exposed to radiolabeled protein at 1 mg/ml. Clones producing antibodies bind a quantity of labeled protein that is detectable above background.

Such clones are expanded and subjected to 2 cycles of cloning at I cell/3 wells. Cloned hybridomas are injected into pristane-treated mice to produce ascites, and monoclonal antibody is purified from the ascitic fluid by affinity chromatography on protein A (APB). Monoclonal antibodies with affinities of at least 10⁸ M⁻¹, preferably 10⁹ to 10¹⁰ M⁻¹ or stronger, are made by procedures well known in the art.

XIII Purification of Naturally Occurring Protein Using Specific Antibodies

Naturally occurring or recombinant protein is substantially purified by immunoaffinity chromatography using antibodies specific for the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIV Screening Molecules for Specific Binding with the cDNA or Protein

The cDNA or fragments thereof and the protein or portions thereof are labeled with ³²P-dCTP, Cy3-dCTP, Cy5-dCTP (APB), or BIODIPY or FITC (Molecular Probes), respectively. Candidate molecules or compounds previously arranged on a substrate are incubated in the presence of labeled nucleic or amino acid. After incubation under conditions for either a cDNA or a protein, the substrate is washed, and any position on the substrate retaining label, which indicates specific binding or complex formation, is assayed. The binding molecule is identified by its arrayed position on the substrate. Data obtained using different concentrations of the nucleic acid or protein are used to calculate affinity between the labeled nucleic acid or protein and the bound molecule. High throughput screening using very small assay volumes and very small amounts of test compound is fully described in Burbaum et al. U.S. Pat. No. 5,876,946.

All patents and publications mentioned in the specification are incorporated herein by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

TABLE 1

| GENBANK ID | DESCRIPTION | BDE |
|---|---|---|
| g1006656 | H. sapiens mRNA for cathepsin C. | 19.3, 17.0, 16.7, 17.1, 14.3 |
| g1262337 | H. sapiens MB1 gene. | 3.9, 6.7 |
| g1280140 | Human eotaxin precursor mRNA, complete cds. | 5.2, 22.6, 6.2, 4.5, 4.4 |
| g1373145 | Human clathrin assembly protein lymphoid myeloid leukemia | 3.7 |
| g1434908 | Homo sapiens MHC (HLA) DRB intron 1 DNA, partial sequence. | 3.4 |
| g1536878 | Human mRNA for chemokine, complete cds. | 5.7, 3.8, 5.3 |
| g178535 | CD13; Human aminopeptidase N | 3.9 |
| g179338 | Human HLA-B-associated transcript 2 (BAT2) mRNA, complete | 6.1 |
| g179640 | Human complement C1q B-chain gene, exon A + 1. | 15.2, 11.1, 8.8, 16.9, 5.3 |
| g180055 | Human thymocyte antigen CD1b mRNA, complete cds. | 5.3, 3.1, 3.2 |
| g180112 | Human antigen CD36 (clone 21) mRNA | 3.3 |
| g180149 | Human transmembrane protein (CD59) gene, exon 4. | 12.9 |
| g180154 | Human lysosomal membrane glycoprotein CD63 mRNA. | 4.0, 10.4 |
| g181179 | Human cathepsin D mRNA, complete cds. | 6.7, 4.6, 9.6 |
| g182489 | Human Fc-epsilon-receptor gamma-chain gene, complete cds. | 4.7, 4.2, 3.6 |
| g182598 | Human IgG low affinity Fc fragment receptor (FcRIIb3) mRNA, | 4.2 |
| g183089 | Human low-affinity Fc-receptor IIB gene, exons 4-7. | 3.1 |
| g184422 | Human 90 kD heat shock protein gene, complete cds. | 5.2 |
| g184851 | Human low affinity IgG receptor CD16 (FcGRIII) mRNA, complete | 3.5, 4.5 |
| g186927 | Homo sapiens lysosomal membrane glycoprotein-1 (LAMP1) mRNA | 7.2 |
| g187054 | Human lymphocyte clathrin light-chain A mRNA, complete cds. | 3.9 |
| g187284 | Human Mac-1 gene encoding complement receptor type 3, CD11b, | 5.1, 4.6, 3.2, 3.1 |
| g187434 | Human monocyte chemotactic and activating factor (MCAF) | 11.1 |
| g187447 | Human monocyte chemotactic protein gene, complete cds. | 15.2, 8.8, 7.3, 12.1, 9.8 |
| g188114 | Human MHC class II HLA-DQ-beta (DQB1, DQw9), complete cds. | 3.3, 3.3 |
| g188134 | Human MHC class II DQ alpha mRNA, complete cds. | 6.6, 4.9, 7.1 |
| g188136 | Human MHC class II HLA-DQ-alpha (DR2-DQw1/DR4 DQw3) mRNA, | 3.4, 3.8 |
| g188217 | Human MHC class II HLA-DR4-beta (DR4,w4) gene, exon 3,4,5,6. | 4.2, 4.8 |
| g188285 | Human MHC class II HLA-DRB4-null beta chain pseudogene | 4.0 |
| g188352 | Human MHC class II HLA-DRw53-associated glycoprotein | 4.0 |
| g188427 | Human HLA-DR alpha-chain (chain-p34) gene, exons 2, 3, 4 | 5.5 |
| g1905800 | H. sapiens MCP-2 gene. | 3.7, 3.3, 7.3 |
| g219907 | Human LD78 beta gene. | 4.9 |
| g264772 | thymosin beta-10 [human, metastatic melanoma cell line, | 3.4 |
| g288396 | H. sapiens NC28 mRNA for monocyte chemoattractant protein | 3.2 |
| g291887 | Homo sapiens cathepsin B mRNA, complete cds. | 31.5, 44.0, 22.1, 5.3, 10.6 |
| g291926 | Homo sapiens cystatin B mRNA, complete cds. | 5.2 |
| g29571 | Homo sapiens Hsc 70 pseudogene. | 6.9, 10.6 |
| g29709 | Human mRNA for cathepsin H (EC 3.4.22.16). | 3.2, 4.2, 3.2 |
| g29714 | Human mRNA for pro-cathepsin L (major excreted protein MEP). | 19.9, 34.8, 29.3, 23.5, 4.3 |
| g29850 | Human CDw40 mRNA for nerve growth factor receptor-related | 4.1, 3.9, 5.0 |
| g3002790 | Homo sapiens macrophage receptor MARCO mRNA, complete cds. | 4.2, 6.0, 7.7 |
| g30257 | human CST3 gene for cystatin C. | 3.9, 3.7, 3.5 |
| g30263 | H. sapiens CST4 gene for Cystatin D. | 3.6, 3.2, 3.9 |
| g30884 | Human mRNA for HLA class II DR-beta 1 (Dw14). | 3.8 |
| g3107914 | Homo sapiens mRNA for cathepsin V, complete cds. | 3.8, 4.5, 16.0 |
| g312143 | H. sapiens mRNA for M130 antigen cytoplasmic variant 1. | 3.4, 3.1 |
| g31335 | Human FcRII mRNA for immunoglobulin G receptor. | 3.6 |
| g31396 | Human mRNA for fibronectin (FN precursor). | 10.0, 76.4, 3.2, 15.0, 81.3, |
| | | 9.6, 19.7, 28.9, 5.6, 27.9 |
| g32210 | Human mRNA for HLA class II DR-beta (HLA-DR B). | 4.3, 5.5 |
| g32291 | Human mRNA fragment for class II histocompatibility antigen | 4.0 |
| g339688 | Human thymosin beta-4 mRNA, complete cds. | 3.8, 4.6 |
| g34511 | Membrane cofactor protein part of complement system | 3.1 |
| g34513 | H. sapiens mRNA for monocyte chemoattractant protein 1 | 11.9, 11.3 |
| g34648 | Human gene for class II invariant gamma-chain (exon 2-8). | 4.4, 4.2, 4.1 |
| g35221 | Human heat-shock protein HSP70B' gene. | 3.3 |
| g3618344 | Homo sapiens mRNA for 26S proteasome subunit p28 | 3.3 |
| g37260 | human tra1 mRNA for human homologue of | 7.7, 7.0 |
| | murine tumor rejection protein | |
| g3776070 | histone deacetylase-like protein and complement component C2 | 3.6, 3.9, 3.8, 3.5 |
| g3869137 | Homo sapiens gene for CC chemokine PARC precursor | 26.4 |
| g3928171 | Human mRNA for beta-Fc-gamma receptor II | 4.1 |
| g4038732 | Homo sapiens mRNA for beta 2-microglobulin, complete cds | 3.1, 3.4 |
| g4097420 | Human monocyte chemotactic protein-4 precursor (MCP-4) | 11.2 |
| g439838 | Human CD86 antigen mRNA, complete cds. | 3.6 |
| g487829 | H. sapiens leukocyte adhesion glycoprotein p150,95 mRNA (CD11c) | 3.5, 4.1 |
| g5478221 | Homo sapiens MHC class II antigen (HLA-DRB1) mRNA, | 4.3, 7.9 |
| g557701 | Human HLA-DMB mRNA, complete cds. | 3.2 |
| g565646 | Human mRNA for proteasome subunit HsC10-II, complete cds. | 3.7 |
| g565648 | Human mRNA for proteasome subunit HsC7-I, complete cds. | 3.5 |
| g5725511 | Homo sapiens beta-2 microglobulin gene, complete cds. | 3.3 |
| g6288733 | Homo sapiens chemokine-like factor 1 (CKLF1) mRNA, | 3.7 |
| g6456875 | Home sapiens thymic stroma chemokine-1 precursor, mRNA, | 5.3 |
| g6467379 | Homo sapiens cathepsin Z precursor (CTSZ) mRNA, complete | 9.0 |
| g662840 | Human heat shock protein 27 (HSPB1) gene exons 1-3, complete | 3.8 |
| g6630853 | Homo sapiens chemokine-like factor 2 (CKLF2) mRNA, | 4.3 |
| g6996445 | Homo sapiens p10 gene for chaperonin 10 (Hsp10 protein) and | 5.0 |
| g703088 | Homo sapiens MHC class II DPw3-alpha-1 chain mRNA, complete | 6.4, 6.0, 5.3 |
| g971269 | Human mRNA for proteasome subunit p40 / Mov34 protein, | 3.1 |
| g188675 | Human mannose receptor mRNA, complete cds. | 47.6, 15.8 |

TABLE 2

| SEQ | | | | | | |
|---|---|---|---|---|---|---|
| ID NO | CLONE ID | TEMPLATE ID | GENBANK ID | DESCRIPTION | DATABASE | BDE |
| 1 | 1728696 | 992917.38 | g762940 | apoferritin H subunit | genpept | −4.4 |
| 2 | 3486093 | 977676.22 | g6103358 | smoothelin-B | genpept | −8.9 |
| 3 | 1563184 | 891492.1 | g1911529 | ficolin [human, uterus, mRNA, 1736 nt]. | gbpri | −4.7 |
| 3 | 1716583 | 891492.1 | g1911529 | ficolin [human, uterus, mRNA, 1736 nt]. | gbpri | −10.1 |
| 4 | 379425 | 990840.1 | g29980 | H. sapiens CL 100 mRNA for protein tyrosine phosphatase | gbpri | −4.4 |
| 5 | 2232685 | 990840.12 | g29980 | H. sapiens CL 100 mRNA for protein tyrosine phosphatase | gbpri | −4.1 |
| 4 | 2232685 | 990840.1 | g29980 | H. sapiens CL 100 mRNA for protein tyrosine phosphatase | | |
| 6 | 1900285 | 021899.7 | g401762 | Homo sapiens ataxia-telangiectasia group D-associated protein | gbpri | −3.4 |
| 7 | 292810 | 203351.6 | g1754608 | Homo sapiens DNA for SCM-1 beta precursor, complete cds. | gbpri | −3.2 |
| 8 | 341491 | 899156.22 | g29903 | Human cellular oncogene c-fos | gbpri | −5, −5.4, −5 |
| 9 | 1425456 | 013003.2 | g5138994 | Homo sapiens mRNA for dual specificity phosphatase MKP-5 | gbpri | −4.1 |
| 10 | 659029 | 237649.2 | g6942243 | Homo sapiens synaptic glycoprotein SC2 (SC2) mRNA | gbpri | −7.8 |
| 11 | 1217764 | 269725.3 | g609453 | Human G0S2 protein gene, complete cds. | gbpri | −3.6 |
| 12 | 2850723 | 989992.6 | g292036 | Human helix-loop-helix basic phosphoprotein (G0S8) gene, | gbpri | −3.3 |
| 13 | 2850723 | 989992.1 | g292036 | Human helix-loop-helix basic phosphoprotein (G0S8) gene, | | |
| 14 | 200836 | 246772.4 | g35791 | Human HPTP epsilon mRNA for protein tyrosine phosphatase | gbpri | −4 |
| 15 | 2197952 | 1327183.6 | g2828536 | Human Hsp27 ERE-TATA-binding protein (HET) mRNA | gbpri | −4.9 |
| 16 | 1958560 | 127112.53 | g292833 | Human TR3 orphan receptor mRNA, complete cds. | gbpri | −3.4 |
| 17 | 1958560 | 127112.19 | | | | |
| 18 | 1506061 | 336965.2 | g186410 | Human inhibin A-subunit mRNA, complete cds. | gbpri | −5.2 |
| 19 | 2172334 | 253946.17 | g186353 | Human membrane glycoprotein gp130 mRNA, complete cds. | gbpri | −5.1 |
| 20 | 1713166 | 284616.2 | g219868 | Human mRNA for HM89. | gbpri | −3.3 |
| 21 | 1640312 | 401269.21 | g404012 | Human pre-B cell enhancing factor (PBEF) | gbpri | −5.7 |
| 22 | 3238201 | 401269.14 | g404012 | Human pre-B cell enhancing factor (PBEF) | gbpri | −3.1, −3.3 |
| 21 | 3238201 | 401269.21 | g404012 | Human pre-B cell enhancing factor (PBEF) | | |
| 23 | 3280041 | 401269.5 | g404012 | Human pre-B cell enhancing factor (PBEF) | gbpri | −5.7 |
| 21 | 3280041 | 401269.21 | g404012 | Human pre-B cell enhancing factor (PBEF) | | |
| 21 | 2964314 | 401269.21 | g404012 | Human pre-B cell enhancing factor (PBEF) | | |
| 24 | 2964314 | 221172.2 | g5523832 | Incyte Unique | gbpri | −3.8 |
| 25 | 3073089 | 055554.1 | g5523832 | Incyte Unique | gbpri | −3.5 |
| 26 | 1360375 | 331567.1 | | Incyte Unique | genpept | −3.4 |
| 27 | 702933 | 010742.1 | | Incyte Unique | gbpri | −4 |
| 28 | 1562722 | 202426.1 | | Incyte Unique | gbpri | −5.7 |
| 28 | 1871721 | 202426.1 | | Incyte Unique | gbpri | −5.3 |
| 29 | 3486126 | 132211.1 | | Incyte Unique | gbpri | −4.3 |

TABLE 3

| SEQ | | | | | | | |
|---|---|---|---|---|---|---|---|
| ID | | TEMPLATE | GENBANK | | DATA- | | |
| NO | CLONE ID | ID | ID | DESCRIPTION | BASE | BDE | E-VALUE |
| 30 | 2456671 | 018414.15 | g1293679 | Human sorting nexin 1 (SNX1) mRNA, complete cds. | gbpri | 4.0 | |
| 31 | 1901407 | 480348.17 | g3152937 | Homo sapiens sorting nexin 2 (SNX2) mRNA, complete cds. | gbpri | 3.7 | |
| 32 | 1659647 | 1135462.6 | g3127052 | Homo sapiens sorting nexin 3 (SNX3) mRNA, complete cds. | gbpri | 4.5 | |
| 33 | 2607957 | 1094479.5 | g1145690 | Human E2 ubiquitin conjugating enzyme UbcH5C | gbpri | 35.2 | |
| 34 | 2762981 | 155345.4 | g4335940 | Homo sapiens leucine aminopeptidase mRNA, complete cds. | gbpri | 6.1 | |
| 35 | 1661952 | 198893.4 | g7800156 | Home sapiens triggering receptor expressed on myeloid cells 2 | gbpri | 8.8 | |
| 35 | 2630138 | 198893.4 | g7800156 | Homo sapiens triggering receptor expressed | gbpri | 6.0 | |
| | | | | on myeloid cells 2 | | | |
| 35 | 2848362 | 198893.4 | g7800156 | Homo sapiens triggering receptor expressed | gbpri | 6.2 | |
| | | | | on myeloid cells 2 | | | |
| 36 | 1452045 | 200569.1 | g435969 | Human metalloproteinase (HME) mRNA, complete cds. | gbpri | 27.2 | |
| 36 | 1553128 | 200569.1 | g435969 | Human metalloproteinase (HME) mRNA, complete cds. | gbpri | 13.8 | |
| 36 | 1563275 | 200569.1 | g435969 | Human metalloproteinase (HME) mRNA, complete cds. | gbpri | 11.5 | |
| 37 | 2240033 | 233706.3 | g2199511 | Homo sapiens beta-3A-adaptin subunit of the AP-3 complex | gbpri | 3.6 | |
| 38 | 1975430 | 234729.3 | g6731234 | Home sapiens myoferlin (MYOF) mRNA | gbpri | 3.4 | |
| 38 | 2642654 | 234729.3 | g6731234 | Home sapiens myoferlin (MYOF) mRNA | gbpri | 3.2 | |
| 39 | 2060308 | 235152.9 | g1698398 | VAT1, an abundant membrane protein of cholinergic | gbpri | 4.4, 3.9 | |
| | | | | synaptic vesicles | | | |
| 40 | 1528052 | 238213.8 | g177204 | Human type IV collagenase mRNA, complete cds. | gbpri | 3.4 | |
| 41 | 2196988 | 242275.7 | g38068 | M.fuscata mRNA for pepsinogen A-2/3. | gbpri | 3.1 | |
| 42 | 3257673 | 257576.34 | g546087 | cytoplasmic antiproteinase = 38 kda intracellular | gbpri | 3.9 | |
| | | | | serine protease inhibitor (serpin) | | | |
| 43 | 2992363 | 343903.1 | g35951 | Human kidney mRNA fragment for renin (aa 105-401). | gbpri | 3.1 | |
| 44 | 2799255 | 348064.3 | g1172085 | Human squamous cell carcinoma antigen (SCCA1) gene | gbpri | 7.5 | |
| 45 | 1601009 | 413969.1 | g2228241 | Home sapiens clone rasi-3 matrix metalloproteinase RASI-1 | gbpri | 3.2 | |
| 46 | 1747756 | 416874.3 | g340159 | Human pro-urokinase mRNA, complete cds. | gbpri | 5.3 | |
| 47 | 2455295 | 437025.25 | g296739 | H. sapiens mRNA for macropain subunit zeta. | gbpri | 3.1 | |
| 48 | 1985367 | 478620.78 | g1890049 | Home sapiens mRNA for cysteine protease, complete cds. | gbpri | 4.7 | |
| 49 | 1274803 | 902393.23 | g189841 | Human prolidase (imidodipeptidase) mRNA, complete cds. | gbpri | 3.1 | |
| 50 | 1393032 | 992309.26 | g338668 | Human testicular angiotensin converting enzyme mRNA | gbpri | 5.2 | |
| 51 | 1404616 | 992309.27 | g178285 | Human angiotensin I-converting enzyme mRNA, gbpri | 3.3 | | |
| | | | | complete cds. | | | |
| 51 | 1427612 | 992309.27 | g178285 | Human angiotensin I-converting enzyme mRNA, | gbpri | 4.0, 3.4 | |
| | | | | complete cds. | | | |
| 52 | 154946 | 153659.2 | g32576 | H. sapiens mRNA for interleukin-1 receptor antagonist. | gbpri | 6.1, 4.6, 4.1 | |
| 52 | 519653 | 153659.2 | g32576 | H. sapiens mRNA for interleukin-1 receptor antagonist. | gbpri | 6.2 | |
| 53 | 1218389 | 242114.46 | g818880 | Pan troglodytes epididymal secretory protein precursor | gbpri | 6.0, 4.7 | |
| 53 | 1875737 | 242114.46 | g818880 | Pan troglodytes epididymal secretory protein precursor | gbpri | 8.6 | |
| 54 | 1446858 | 242114.47 | g818880 | Pan troglodytes epididymal secretory protein precursor | gbpri | 5.0, 4.8 | |
| 55 | 1375759 | 242114.48 | g818880 | Pan troglodytes epididymal secretory protein precursor | gbpri | 3.4 | |
| 56 | 2722128 | 244973.9 | g2978559 | Homo sapiens vascular endothelial cell growth factor 165 | gbpri | 5.9 | |
| 57 | 1273066 | 290224.2 | g1223889 | Human putative T1/ST2 receptor binding protein precursor | gbpri | 3.1 | |
| 58 | 1314322 | 331428.2 | g1885307 | H. sapiens mRNA for IL13 receptor alpha-1 chain. | gbpri | 3.5 | |
| 59 | 2057240 | 401593.10 | g1203964 | Home sapiens bone-derived growth factor (BPGF-1) | gbpri | 3.1 | |
| 60 | 504786 | 199772.6 | g187297 | Human MAL protein gene mRNA, complete cds | gbpri | 3.4 | |
| 61 | 1505813 | 330836.1 | g189263 | Human notch group protein (N) mRNA, partial cds. | gbpri | 9.9 | |
| 62 | 518094 | 902142.11 | g2653870 | Home sapiens leucocyte immunoglobulin-like receptor-5 | gbpri | 3.1 | |
| 63 | 351384 | 995211.5 | g184428 | Human heparan sulfate proteoglycan (HSPG) core protein, 3′ | gbpri | 6.2, 5.4 | |
| 63 | 1601035 | 995211.5 | g184428 | Human heparan sulfate proteoglycan (HSPG) core protein, 3′ | gbpri | 6.8 | |
| 64 | 1435222 | 235310.12 | g2905995 | Homo sapiens DAP12 gene, complete cds. | gbpri | 4.4 | |
| 65 | 1402833 | 900031.4 | g2094872 | H. sapiens DAP-kinase mRNA. | gbpri | 3.7 | |
| 66 | 2493520 | 013521.17 | g38274 | human ret proto-oncogene mRNA for tyrosine kinase | gbpri | 4.5 | |
| 67 | | 013521.18 | | | | | |
| 68 | 1867423 | 032942.8 | g763112 | H. sapiens ERK3 mRNA. | gbpri | 3.6 | |
| 69 | 157088 | 149914.21 | g1418933 | H. sapiens mRNA for protein-tyrosine-phosphatase | gbpri | 3.1 | |
| 70 | 2501777 | 276948.4 | g31197 | Human c-erb-B-2 mRNA. | gbpri | 5.7 | |
| 71 | 810002 | 347283.16 | g531819 | Human B lymphocyte serine/threonine protein kinase | gbpri | 30.4 | |
| 72 | 1418741 | 349727.2 | g306804 | Human G protein-coupled receptor kinase (GRK5) | gbpri | 19.5 | |
| 73 | 2662471 | 350230.2 | g189982 | Human testis-specific cAMP-dependent protein kinase | gbpri | 3.7 | |
| 74 | 1524551 | 386989.40 | g1526989 | Human cAMP-dependent protein kinase type I-alpha subunit | gbpri | 3.2 | |
| 75 | 1437116 | 434608.8 | g34275 | Human mRNA for T200 leukocyte common | gbpri | 3.2 | |
| | | | | antigen (CD45, LC-A). | | | |
| 76 | 1725791 | 891141.5 | g3043926 | Home sapiens Tat-interacting protein TIP30 | gbpri | 3.1 | |
| 77 | 1820994 | 233653.13 | g2738496 | Homo sapiens putative oral tumor suppressor protein (doc-1) | gbpri | 3.5 | |
| 78 | 191334 | 253405.7 | g1297329 | Human mitogen-responsive phosphoprotein DOC-2 mRNA | gbpri | 5.1 | |
| 79 | 1509469 | 197600.12 | g3800808 | Homo sapiens methyl-CpG binding | gbpri | 3.7 | |
| | | | | protein MBD4 (MBD4) mRNA, | | | |
| 80 | 818223 | 1078199.1 | g38031 | Human ZNF43 mRNA | gbpri | 3.5 | |
| 81 | 2208461 | 025138.1 | g2088550 | similar to 52kd Ro/SSA protein and RFP | gbpri | 9.7 | |
| 82 | 33091 | 1000222.1 | g181485 | Human DNA-binding protein B (dbpB) gene, 3′end. | gbpri | 3.9 | |
| 83 | 1818802 | 1079467.1 | g468707 | H. sapiens OZF mRNA. | gbpri | 3.3 | |
| 84 | 2881835 | 1082203.1 | g6409344 | Home sapiens zinc finger protein ZNF180 (ZNF180) mRNA, | gbpri | 3.1 | |
| 85 | 1434547 | 1096748.6 | g509777 | H. sapiens ERF-2 mRNA. | gbpri | 7.7 | |
| 86 | 1467384 | 1098409.1 | g181986 | Human early growth response 2 protein | gbpri | 3.3 | |
| | | | | (EGR2) mRNA, complete | | | |
| 86 | 3603037 | 1098409.1 | g181986 | Human early growth response 2 protein | gbpri | 3.2 | |
| | | | | (EGR2) mRNA, complete | | | |
| 87 | 2115028 | 1098584.1 | g6644296 | Home sapiens IFI16b (IFI16b) mRNA, | gbpri | 3.5 | |
| | | | | complete cds. | | | |
| 88 | 2115028 | 40652.29 | | | | | |
| 89 | 1685409 | 1099277.17 | g3641537 | Homo sapiens small EDRK-rich factor 2 (SERF2) mRNA | gbpri | 3.9 | |
| 90 | 2735724 | 197748.7 | g5757883 | Home sapiens zinc finger Homeobox protein ZHX1 mRNA, | gbpri | 29.3 | |
| 91 | 1797396 | 241930.15 | g726512 | Human nuclear orphan receptor LXR-alpha | gbpri | 3.1 | |
| | | | | mRNA, complete cds. | | | |
| 92 | 1274720 | 246504.1 | g287642 | H sapiens cDNA for TREB protein. | gbpri | 4.2 | |
| 93 | 1575463 | 253341.11 | g2947300 | Homo sapiens MMS2 (MMS2) mRNA, complete cds. | gbpri | 3.1 | |
| 94 | 1292502 | 334064.3 | g2190973 | Homo sapiens breast cancer suppressor candidate 1 (bcsc-1) | gbpri | 4.9 | |
| 94 | 2378649 | 334064.3 | g2190973 | Home sapiens breast cancer suppressor candidate 1 (bcsc-1) | gbpri | 5.5 | |
| 94 | 2595814 | 334064.3 | g2190973 | Homo sapiens breast cancer suppressor candidate 1 (bcsc-1) | gbpri | 3.6 | |
| 95 | 1554495 | 347723.6 | g1374697 | Homo sapiens mRNA for nuclear protein, | gbpri | 5, 3.4 | |
| | | | | NP220, complete cds. | | | |
| 96 | 1628271 | 902614.15 | g1480099 | H. sapiens mRNA for peroxisome proliferactor activated | gbpri | 4.3 | |
| 97 | 1969563 | 995119.1 | g186624 | Human c-jun proto oncogene (JUN), | gbpri | 4.0 | |
| | | | | complete cds, clone hCJ-1. | | | |
| 98 | 3117417 | 999437.3 | 9498722 | H. sapiens HZF2 mRNA for zinc finger protein from U-937 | gbpri | 3.7 | |
| 99 | 27775 | 1225522.1 | g28434 | Human mRNA for apoferritin H chain type. | gbpri | 3.4, 3.5 | |
| 100 | 262983 | 235447.5 | g37432 | Human mRNA for transferrin receptor. | gbpri | 4.3, 3.3 | |
| 101 | 3121380 | 139899.22 | g182513 | Human ferritin L chain mRNA, complete cds. | gbpri | 23.2 | |
| 102 | 2055571 | 977615.8 | g313158 | H. sapiens mRNA for GM2 activator protein. | gbpri | 4.1 | |
| 103 | 1556827 | 995174.7 | g2276462 | Homo sapiens Niemann-Pick C disease | gbpri | 8.0 | |
| | | | | protein (NPC1) mRNA; similar to patche | | | |
| 104 | 2515389 | 346599.14 | g178848 | Human apolipoprotein E | gbpri | 5.7, 22.3, | |
| | | | | | | 18.8 | |
| 104 | 2516686 | 346599.14 | g178848 | Human apolipoprotein E | gbpri | 17.2, 16.2 | |
| 105 | 2369312 | 1328674.2 | g178830 | Human apolipoprotein C-I (VLDL) gene, complete cds. | gbpri | 3.9 | |
| 106 | 1214221 | 1099498.9 | g178830 | Human apolipoprotein C-I (VLDL) gene, complete cds. | gbpri | 3.4 | |
| 106 | 2604020 | 1099498.9 | g178830 | Human apolipoprotein C-I (VLDL) gene, complete cds. | gbpri | 4.8, 4.0 | |
| 107 | 1694113 | 049457.6 | g187209 | Human lipoprotein lipase mRNA, complete cds. | gbpri | 6.1 | |
| 107 | 2118094 | 049457.6 | g187209 | Human lipoprotein lipase mRNA, complete cds. | gbpri | 4.5 | |
| 108 | 487539 | 092511.1 | g1255603 | Human mRNA for cytosolic malate | gbpri | 3.4 | |
| | | | | dehydrogenase, complete cds. | | | |
| 108 | 491360 | 092511.1 | g1255603 | Human mRNA for cytosolic malate | gbpri | 7.5 | |
| | | | | dehydrogenase, complete cds. | | | |
| 109 | 2446571 | 092511.6 | g1255603 | Human mRNA for cytosolic malate | gbpri | 19.4 | |
| | | | | dehydrogenase, complete cds. | | | |
| 110 | 1488759 | 238609.2 | g1060902 | Human mRNA for phosphatidylinositol transfer protein | gbpri | 4.5 | |
| 110 | 2122844 | 238609.2 | g1060902 | Human mRNA for phosphatidylinositol transfer protein | gbpri | 4.0 | |
| 111 | 2224733 | 252895.15 | g181477 | Human diazepam binding inhibitor (DBI) mRNA | gbpri | 3.3 | |
| 112 | 2805736 | 252895.16 | g181477 | Human diazepam binding inhibitor (DBI) mRNA | gbpri | 4.8 | |
| 113 | 2537805 | 99588012 | g182353 | Human fatty acid binding protein homologue | gbpri | 10.3, 17.5 | |
| | | | | (PA-FABP) mRNA, | | | |
| 114 | 2673842 | 001726.1 | g5901760 | fatty acid binding protein homolog | gbpri | 28.3 | |
| 115 | 4013105 | 179368.2 | g1377853 | Human fatty acid binding protein FABP gene, complete cds. | gbpri | 3.5 | |
| 116 | 1524345 | 410917.7 | g506430 | H. sapiens (HepG2) LAL mRNA for lysosomal acid lipase. | gbpri | 11.5 | |
| 116 | 1970427 | 410917.7 | g506430 | H. sapiens (HepG2) LAL mRNA for lysosomal acid lipase. | gbpri | 6.1 | |
| 117 | 1658320 | 336012.15 | g179459 | Human alpha-polypeptide of N-acetyl-alpha-glucosaminidase | gbpri | 5.8 | |
| 117 | 2448129 | 336012.15 | g179459 | Human alpha-polypeptide of N-acetyl-alpha-glucosaminidase | gbpri | 5.4, 4.6 | |
| 118 | 2096385 | 235128.9 | g1160966 | Homo sapiens palmitoyl-protein thioesterase gene, complete | gbpri | 4.5 | |
| 118 | 1536287 | 235128.9 | g1160966 | Homo sapiens palmitoyl-protein thioesterase gene, complete | gbpri | 5.1 | |
| 119 | 1968372 | 215481.15 | g34733 | Human mRNA for acid phosphatase type 5 (EC 3.1.3.2). | gbpri | 4.9 | |
| 119 | 2206828 | 215481.15 | g34733 | Human mRNA for acid phosphatase type 5 (EC 3.1.3.2). | gbpri | 6.0 | |
| 120 | 3223710 | 885713.30 | g1008454 | Human mitochondrial ATP synthase subunit 9, P3 gene copy, | gbpri | 4.6 | |
| 121 | 3029307 | 902700.6 | g28939 | Human mRNA for F1-ATPase beta subunit (F-1 beta). | gbpri | 3.5 | |
| 122 | 2510502 | 401575.10 | g3335127 | Homo sapiens F1Fo-ATPase synthase | gbpri | 4.1 | |
| | | | | f subunit mRNA, complete | | | |
| 122 | 2816429 | 401575.10 | g3335127 | Homo sapiens F1Fo-ATPase synthase | gbpri | 4.1, 3.6 | |
| | | | | f subunit mRNA, complete | | | |
| 123 | 2487243 | 232818.15 | g3329377 | Homo sapiens vacuolar H(+)-ATPase | gbpri | 3.4 | |
| | | | | subunit mRNA, complete | | | |
| 124 | 921297 | 233103.10 | g1395161 | Homo sapiens mRNA for vacuolar ATPase, complete cds. | gbpri | 3.5 | |
| 124 | 2192978 | 233103.10 | g1395161 | Homo sapiens mRNA for vacuolar ATPase, complete cds. | gbpri | 3.9 | |
| 125 | 2510187 | 234313.13 | g509290 | H. sapiens mRNA for H+-ATP synthase subunit b. | gbpri | 3.1 | |
| 126 | 2510187 | 234313.14 | | | | | |
| 126 | 2683449 | 234313.14 | g509290 | H. sapiens mRNA for H+-ATP synthase subunit b. | gbpri | 3.8 | |
| 127 | 1648713 | 246862.17 | g559324 | Human mRNA for ATP synthase alpha subunit, complete cds. | gbpri | 6.2, 6.0 | |
| 128 | 859321 | 253969.2 | g522192 | Homo sapiens vacuolar H+-ATPase | gbpri | 12.5 | |
| | | | | Mr 56,000 subunit (HO57) | | | |
| 128 | 2075603 | 253969.2 | g522192 | Homo sapiens vacuolar H+-ATPase | gbpri | 7.5 | |
| | | | | Mr 56,000 subunit (HO57) | | | |
| 129 | 1232362 | 328615.1 | g2393721 | Human glutathione-S-transferase homolog | gbpri | 5.0 | |
| | | | | mRNA, complete cds. | | | |
| 130 | 2708221 | 998594.2 | g1747520 | Homo sapiens microsomal glutathione S-transferase 2 | gbpri | 3.1 | |
| 131 | 1923262 | 220837.3 | g183141 | Human gamma-glutmyl transpeptidase-related | gbpri | 3.2 | |
| | | | | protein (GGT-Rel) | | | |
| 132 | 2099268 | 410296.1 | g4106439 | multidrug resistance-associated protein 3 | gbpri | 5.1 | |
| 132 | 2642164 | 410296.1 | g3550323 | Homo sapiens canalicular multispecific | gbpri | 4.6, 4.3 | |
| | | | | organic anion transporter; similar to MRP | | | |
| 133 | 851304 | 218849.9 | g5006263 | g5006263 organic anion transporter OATP-B | genpept | 4.6 | |
| 134 | 851304 | 218849.15 | | | | | |
| 135 | 374690 | 218849.16 | g5006263 | g5006263 organic anion transporter OATP-B | genpept | 4.1 | |
| 133 | 374690 | 218849.9 | | | | | |
| 135 | 1512278 | 218849.16 | g5006263 | g5006263 organic anion transporter OATP-B | genpept | 5.0 | |
| 136 | 2448113 | 233319.14 | g3329393 | Homo sapiens voltage dependent | gbpri | 4.0 | |
| | | | | anion channel protein mRNA, | | | |
| 137 | 2021456 | 434763.22 | g340198 | Human voltage-dependent anion channel | gbpri | 3.3 | |
| | | | | isoform 1 (VDAC) mRNA, | | | |
| 138 | 2021456 | 434763.18 | | | | | |
| 139 | 1269859 | 336954.1 | g2739502 | Homo sapiens potassium channel mRNA, | gbpri | 4.7 | |
| | | | | complete cds. | | | |
| 140 | 2742236 | 337928.1 | g2584784 | H. sapiens mRNA homologous to the p64 | gbpri | 3.2 | |
| | | | | bovine chloride channel | | | |
| 141 | 109018 | 430431.18 | g4426566 | Homo sapiens chloride channel ABP mRNA, complete cds. | gbpri | 3.4 | |
| 142 | 2361578 | 407376.3 | g4581469 | Homo sapiens mRNA for SLC7A8 protein. | gbpri | 3.9 | |
| 143 | 1749790 | 439010.7 | g6826913 | Homo sapiens calcium transport ATPase ATP2C1 | gbpri | 4.5 | |
| 144 | 2056395 | 245184.1 | g339567 | Human transforming growth factor-beta induced gene product | gbpri | 10.4, 11.0 | |
| 145 | 2965657 | 352406.29 | g3360431 | Homo sapiens clone 23810 osteopontin mRNA, complete cds. | gbpri | 28.5 | |
| 146 | 229442 | 352406.28 | g3360431 | Homo sapiens clone 23810 osteopontin mRNA, complete cds. | gbpri | 11.5 | |
| 147 | 2495131 | 992455.45 | g187109 | Human 14 kd lectin mRNA, complete cds. | gbpri | 5.5 | |
| 148 | 2057608 | 255957.1 | g4323580 | Homo sapiens senescence-associated epithelial membrane | gbpri | 14.4 | |
| 149 | 1310287 | 235164.25 | g28525 | Human mRNA for amyloid A4 precursor | gbpri | 3.2 | |
| | | | | of Alzheimer's disease. | | | |
| 150 | 2645263 | 997363.3 | g4586835 | Homo sapiens mRNA for type II membrane protein | gbpri | 3.3 | |
| 151 | | 997363.1 | | | | | |
| 152 | 853968 | 1327354.1 | g32014 | Human HALPHA44 gene for alpha-tubulin, exons 1-3. | gbpri | 3.6 | |
| 152 | 1296056 | 1327354.1 | g32014 | Human HALPHA44 gene for alpha-tubulin, exons 1-3. | gbpri | 4.1 | |
| 153 | 1522716 | 358853.44 | g37851 | H. sapiens vimentin gene. | gbpri | 10.1 | |
| 154 | 2508570 | 243019.14 | g187455 | Homo sapiens macrophage capping protein | gbpri | 3.4 | |
| | | | | mRNA, complete cds. | | | |
| 155 | 3208407 | 1000016.22 | g36502 | Human mRNA for enteric smooth muscle gamma-actin. | gbpri | 4.0 | |
| 156 | | 1000016.44 | | | | | |
| 157 | 218631 | 1000084.41 | g340020 | human alpha-tubulin mRNA, complete cds. | gbpri | 6.1 | |
| 158 | 1269670 | 481480.2 | g7012928 | Homo sapiens SCG10 like-protein | gbpri | 3.5 | |
| 159 | 1469939 | 214181.1 | g461396 | Homo sapiens alpha-1 type XV collagen mRNA, | gbpri | 3.6 | |
| | | | | complete cds. | | | |
| 160 | 2190217 | 977305.3 | g829622 | Human myosin regulatory light chain mRNA, complete cds. | gbpri | 4.5, 3.3 | |
| 161 | 2379695 | 450223.3 | g7288158 | Homo sapiens mRNA for testis specific ankyrin-like protein | gbpri | 6.0 | |
| 162 | 2665489 | 375072.25 | g2739095 | Homo sapiens protein 4.1-G mRNA, complete cds. | gbpri | 3.1 | |
| 163 | 2926914 | 1039889.12 | g28251 | Human mRNA for beta-actin. | gbpri | 4.0 | |
| 164 | 3215205 | 245367.2 | g5932031 | Homo sapiens PEA15 protein (PEA15) gene, | 4.1 | | |
| | | | | exons 3 and 4 and | gbpri | | |
| 165 | 2444446 | 252747.21 | g998356 | Homo sapiens EB1 mRNA, complete cds | gbpri | 3.8 | |
| 166 | 1648449 | 229250.3 | g3006201 | Homo sapiens prostaglandin transporter | gbpri | 14.1 | |
| | | | | hPGT mRNA, complete | | | |
| 167 | 1886871 | 331734.3 | g189886 | Human prostaglandin endoperoxide | gbpri | 3.4 | |
| | | | | synthase mRNA, complete cds. | | | |
| 167 | 1595081 | 331734.3 | g249623 | prostaglandin G/H synthase {alternative splicing product} | gbpri | 4.7 | |
| 168 | | 331734.8 | | | | | |
| 169 | 1401683 | 441261.5 | g862374 | Human 180 kDa transmembrane PLA2 | gbpri | 4.6 | |
| | | | | receptor mRNA, complete cds. | | | |
| 170 | 638749 | 411208.4 | g2911111 | Homo sapiens mRNA for NRAMP2; innate immunity | gbpri | 3.4 | |
| 170 | 933140 | 411208.4 | g2911111 | Homo sapiens mRNA for NRAMP2; innate immunity | gbpri | 3.1, 3.1 | |
| 171 | 1241477 | 335940.2 | g2459617 | Homo sapiens Toll-like receptor 1 | gbpri | 3.4 | |
| | | | | (TLR1) mRNA, complete cds. | | | |
| 172 | 1661537 | 369213.26 | g177869 | Human alpha-2-macroglobulin mRNA, complete cds. | gbpri | 13.3 | |
| 173 | 1605616 | 222156.2 | g1050957 | Human chitotriosidase precursor mRNA, complete cds. | gbpri | 9.7 | |
| 174 | 1000988 | 234404.4 | g182836 | Human factor XIII subunit a mRNA, 3′ end. | gbpri | 11.0 | |
| 174 | 1874280 | 234404.4 | g182836 | Human factor XIII subunit a mRNA, 3′ end. | gbpri | 11.9, 9.3 | |
| 175 | 1436753 | 474310.15 | g339520 | Human transglutaminase (TGase) mRNA, complete cds. | gbpri | 22.7 | |
| 175 | 1672744 | 474310.15 | g339520 | Human transglutaminase (TGase) mRNA, complete cds. | gbpri | 14.5 | |
| 176 | 1557811 | 254107.1 | g37123 | Human mRNA for thrombomodulin precursor. | gbpri | 4.3 | |
| 177 | 1220371 | 218156.2 | g3925598 | Homo sapiens mRNA for DORA protein | gbpri | 14.5 | |
| 178 | 1533410 | 350788.2 | g407955 | Human membrane-associated protein (HEM-1) | gbpri | 4.7 | |
| | | | | mRNA, complete cds. | | | |
| 179 | 1999422 | 899239.5 | g29645 | H. sapiens mRNA for CAMPATH-1 (CDw52) antigen. | gbpri | 4.2, 3.5 | |
| 180 | 322866 | 382906.6 | g180142 | Human CD53 glycoprotein mRNA, complete cds. | gbpri | 3.2 | |
| 181 | 1874037 | 382906.16 | g180142 | Human CD53 glycoprotein mRNA, complete cds. | gbpri | 19.5 | |
| 182 | 2419118 | 269837.15 | g6563293 | Homo sapiens PIL protein mRNA, complete cds. | gbpri | 3.5 | |
| 183 | 1689409 | 241016.6 | g1737489 | Human p76 mRNA, complete cds. Membrane protein | genpept | 4.6 | |
| 184 | 2182130 | 005486.4 | g4336324 | Homo sapiens small membrane protein 1 (SMP1) mRNA, | genpept | 3.2 | |
| 185 | 2242283 | 977953.8 | | Incyte Unique | | 4.5 | |
| 186 | 2041671 | 404923.1 | | Incyte Unique | | 5.5 | |
| 187 | 1621280 | 449028.32 | | Incyte Unique | | 3.1 | |
| 188 | 1869766 | 330864.4 | | Incyte Unique | | 4.6 | |
| 189 | 1427838 | 39903.4 | | Incyte Unique | | 3.7, 3.2 | |
| 190 | 1683829 | 250445.3 | | Incyte Unique | | 4.0 | |
| 191 | 1996005 | 400961.22 | | Incyte Unique | | 3.3 | |
| 192 | 2503016 | 977835.14 | | Incyte Unique | | 3.1 | |
| 193 | 1748678 | 252920.1 | | Incyte Unique | | 4.7 | |
| 194 | 2716391 | 343863.10 | | Incyte Unique | | 3.1 | |
| 195 | 3134070 | 254547.9 | | Incyte Unique | | 10.3 | |
| 196 | 1996357 | 277726.11 | | Incyte Unique | | 4.1, 3.8 | |
| 197 | 1999313 | 246177.4 | | Incyte Unique | | 4.8 | |
| 198 | 2046715 | 410012.21 | | Incyte Unique | | 5.7 | |
| 199 | 1886544 | 410012.24 | | Incyte Unique | | 4.4 | |
| 200 | | 410012.20 | | Incyte Unique | | | |
| 201 | 2628690 | 1099099.16 | g2772672 | phospholipase D-like protein; g1575347 HU-K4 | genpept | 4.5 | 3.00E−99 |
| 202 | | 1099099.13 | | | | | |
| 201 | 642116 | 1099099.16 | g2772672 | phospholipase D-like protein; g1575347 HU-K4 | genpept | 3.7, 3.2 | |
| 203 | 663537 | 400253.14 | g2826476 | IL-17 receptor | genpept | 8.5 | 9.00E−30 |
| 204 | | 400253.17 | | | | | |
| 205 | 1509631 | 85803.4 | g50229 | C1q C chain [Mus musculus] | genpept | 9.1, 7.3 | 3.00E−71 |
| 206 | 2790509 | 253868.2 | g3879017 | similar to Ubiquitin-conjugating enzymes; cDNA EST | genpept | 3.5 | 2.00E−42 |
| 206 | 1426987 | 253868.2 | g3879017 | similar to Ubiquitin-conjugating enzymes; cDNA EST | genpept | 3.6 | 2.00E−42 |
| 207 | 2674742 | 158923.9 | g607003 | beta transducin-like protein | genpept | 4.2 | 2.00E−29 |
| 208 | 3330716 | 199716.3 | g7243708 | zinc transporter like 1 [Mus musculus] | genpept | 3.2 | 0 |
| 209 | 1620875 | 252899.8 | g6176319 | butyrophilin-like [Homo sapiens ] | genpept | 3.2 | 4.00E−35 |
| 210 | 627910 | 327544.2 | g7406950 | N system amino acids transporter NAT-1 [Mus musculus] | genpept | 5.0 | 1.00E−112 |
| 210 | 2007779 | 327544.2 | g7406950 | N system amino acids transporter NAT-1 [Mus musculus] | genpept | 6.1 | 1.00E−112 |
| 210 | 2726053 | 327544.2 | g7406950 | N system amino acids transporter NAT-1 [Mus musculus] | genpept | 4.6, 3.9 | 1.00E−112 |
| 211 | 868138 | 334842.5 | g6175860 | g1-related zinc finger protein [Mus musculus] | genpept | 22.7 | 2.00E−84 |
| 212 | 938514 | 406770.1 | g1841756 | GATA-5 cardiac transcription factor [Mus musculus] | genpept | 3.2 | 2.00E−84 |
| 213 | 518335 | 996806.15 | g19867 | extensin (AA 1-620) | genpept | 3.1 | 0.018 |
| 213 | 2727735 | 996806.15 | g19867 | extensin (AA 1-620) | genpept | 4.1 | 0.018 |
| 214 | 2495063 | 332263.1 | g5817244 | dJ20N2.1 similar to yeast and bacterial cytosine deaminase | genpept | 4.1 | 1.00E−89 |
| 215 | 2659055 | 255824.34 | g6572231 | dJ402G11.6 similar to Xenopus gamma-tubulin | genpept | 4.0 | 0 |
| 216 | 1963927 | 1095059.19 | | Incyte Unique | | 3.5 | |
| 217 | 1662589 | 902753.4 | | Incyte Unique | | 4.6 | |
| 218 | 2674037 | 232259.1 | | Incyte Unique | | 4.0 | |
| 219 | 1220608 | 330879.1 | | Incyte Unique | | 3.2 | |
| 220 | 1868520 | 370489.34 | | Incyte Unique | | 3.5 | |
| 221 | 1447681 | 233007.5 | | Incyte Unique | | 3.3 | |
| 222 | 2236427 | 422389.9 | | Incyte Unique | | 3.1 | |
| 223 | 1632936 | 228814.8 | | Incyte Unique | | 3.4 | |
| 223 | 1519414 | 228814.8 | | Incyte Unique | | 3.1 | |
| 224 | | 228814.10 | | Incyte Unique | | | |
| 225 | 519879 | 234169.3 | | Incyte Unique | | 3.4 | |
| 226 | 1656490 | 403676.1 | | Incyte Unique | | 15.9 | |
| 227 | 1536275 | 231546.1 | | Incyte Unique | | 3.3 | |
| 228 | 2212027 | 247544.1 | | Incyte Unique | | 7.2 | |
| 229 | 782704 | 237320.1 | | Incyte Unique | | 3.2 | |
| 230 | 1848664 | 983197.1 | | Incyte Unique | | 13.3 | |
| 231 | 777478 | 481455.4 | | Incyte Unique | | 3.6 | |
| 231 | 2444774 | 481455.4 | | Incyte Unique | | 4.2 | |
| 232 | | 481455.6 | | | | | |
| 233 | 2718363 | 237733.2 | | Incyte Unique | | 3.3 | |
| 234 | 1907948 | 040151.2 | | Incyte Unique | | 3.4 | |
| 235 | 1864720 | 425448.2 | | Incyte Unique | | 3.6 | |
| 236 | 1857223 | 012712.1 | | Incyte Unique | | 4.2 | |
| 237 | 104201 | 009707.3 | | Incyte Unique | | 4.7 | |
| 238 | 1871285 | 019751.1 | | Incyte Unique | | 3.9 | |
| 239 | 1931160 | 065689.1 | | Incyte Unique | | 12.7 | |
| 240 | 1611844 | 279164.6 | | Incyte Unique | | 3.1 | |
| 241 | 2041593 | 428329.1 | | Incyte Unique | | 3.2 | |

TABLE 4

| SEQ | | | | | | |
|---|---|---|---|---|---|---|
| ID | | | | | | |
| NO | CLONE ID | TEMPLATE ID | GENBANK ID | DESCRIPTION | DATABASE | BDE |
| 242 | 154371 | 988891.1 | g35662 | Human mRNA for prointerleukin 1 beta. | gbpri | −3.1 |
| 243 | | 988891.13 | | | | |
| 244 | 3661567 | 1327819.1 | g306500 | Human CD9 antigen mRNA, complete cds; splice variant? | gbpri | −3.1 |
| 245 | | 1328161.1 | | | | |
| 246 | 1904149 | 1099039.5 | g307390 | Human ribosomal protein S13 (RPS13) mRNA, complete cds. | gbpri | −4.2 |
| 247 | 2527879 | 1328423.2 | g682747 | Human mRNA for Apo1_Human (MER5(Aop1-Mouse)-like protein), | gbpri | −2.8 |
| 248 | 3168108 | 232691.21 | g1518017 | Human TRAF-interacting protein I-TRAF mRNA, complete cds. | gbpri | −3.2 |
| 249 | 2785701 | 241888.40 | g186367 | Human interleukin 8 (IL8) gene, complete cds. | gbpri | −8.4 |
| 249 | 3732536 | 241888.40 | g186367 | Human interleukin 8 (IL8) gene, complete cds. | gbpri | −12.5 |
| 250 | 1972768 | 256005.49 | g178017 | Human activation (Act-2) mRNA, complete cds. | gbpri | −3.3 |
| 251 | 80154 | 256005.51 | g178017 | Human activation (Act-2) mRNA, complete cds. | gbpri | −4.6 |
| 252 | 1618459 | 331851.5 | g2276318 | Homo sapiens mRNA for dynein heavy chain. | gbpri | −2.8 |
| 253 | 1603857 | 341546.3 | g3978243 | Homo sapiens inhibitor of apoptosis protein-1 (MIHC) mRNA, | gbpri | −3 |
| 254 | 3126072 | 400834.2 | g5042402 | UCRI_HUMAN; RIESKE IRON-SULFUR PROTEIN; RISP | genpept | −2.6 |
| 255 | 4718119 | 407740.1 | g5305442 | Homo sapiens carnitine octanoyltransferase mRNA, complete | gbpri | −5 |
| 256 | 1543740 | 1082684.1 | | Incyte Unique | | −4.5 |
| 257 | 3122044 | 318000.8 | | Incyte Unique | | −3 |

TABLE 5

| SEQ ID NO | CLONE ID | TEMPLATE ID | GENBANK ID | DESCRIPTION | DATABASE | BDE |
|---|---|---|---|---|---|---|
| 258 | 1943863 | 206382.35 | g2558583 | neurogranin | genpept | 4 |
| 259 | 790977 | 1090400.1 | | Incyte Unique | | 2.9 |
| 260 | 1444550 | 018342.1 | | Incyte Unique | | 2.6 |

What is claimed is:

1. A combination comprising a plurality of cDNAs that are differentially expressed in DC, and selected from SEQ ID NOs:1-260 or their complements.

2. The combination of claim 1, wherein each of the cDNAs is downregulated in DC and is selected from SEQ ID NOs:1-29.

3. The combination of claim 1, wherein each of the cDNAs is upregulated in DC and is selected from SEQ ID NOs:30-241.

4. The combination of claim 1, wherein each of the cDNAs is upregulated in mature DC and is selected from SEQ ID NOs:242-257.

5. The combination of claim 1, wherein each of the cDNAs is downregulated in mature DC and is selected from SEQ ID NOs:258-260.

6. The combination of claim 1, wherein the cDNAs are immobilized on a substrate.

7. A high throughput method for detecting differential expression of one or more cDNAs in a sample containing nucleic acids, the method comprising:
(a) hybridizing the substrate of claim 6 with nucleic acids of the sample, thereby forming one or more hybridization complexes;
(b) detecting the hybridization complexes; and
(c) comparing the hybridization complexes with those of a standard, wherein differences between the standard and sample hybridization complexes indicate differential expression of cDNAs in the sample.

8. The method of claim 7, wherein the nucleic acids of the sample are amplified prior to hybridization.

9. The method of claim 7, wherein the sample is from a subject with cancer, infectious disease, autoimmunity, allergy, or graft versus host disease and comparison with a standard identifies or stages the disorder.

10. A high throughput method of screening a plurality of molecules or compounds to identify a ligand which specifically binds a cDNA, the method comprising:
(a) combining the combination of claim 1 with the plurality of molecules or compounds under conditions to allow specific binding; and
(b) detecting specific binding between each cDNA and at least one molecule or compound, thereby identifying a ligand that specifically binds to each cDNA.

11. The method of claim 10 wherein the plurality of molecules or compounds are selected from aptamers, DNA molecules, RNA molecules, peptide nucleic acid molecules, mimetics, peptides, transcription factors, repressors, and regulatory proteins.

12. An isolated cDNA selected from the group consisting of SEQ ID NOs:24-29, 187-202, 205-213, 216-241, 256, 257, 259, and 260.

13. A vector containing the cDNA of claim 12.

14. A host cell containing the vector of claim 13.

15. A method for producing a protein, the method comprising the steps of:
(a) culturing the host cell of claim 14 under conditions for expression of protein; and
(b) recovering the protein from the host cell culture.

16. A protein or a portion thereof produced by the method of claim 15.

17. A high-throughput method for using a protein to screen a plurality of molecules or compounds to identify at least one ligand which specifically binds the protein, the method comprising:
(a) combining the protein of claim 16 with the plurality of molecules or compounds under conditions to allow specific binding; and
(b) detecting specific binding between the protein and a molecule or compound, thereby identifying a ligand which specifically binds the protein.

18. The method of claim 17 wherein the plurality of molecules or compounds is selected from DNA molecules, RNA molecules, peptide nucleic acid molecules, mimetics, peptides, proteins, agonists, antagonists, antibodies or their fragments, immunoglobulins, inhibitors, drug compounds, and pharmaceutical agents.

19. A method of using a protein to produce an antibody, the method comprising:
a) immunizing an animal with the protein of claim 16 under conditions to elicit an antibody response;
b) isolating animal antibodies; and
c) screening the isolated antibodies with the protein, thereby identifying an antibody which specifically binds the protein.

20. A antibody produced by the method of claim 19.
